SEQUENCE LISTING (8 sequences)

SEQ ID NO: 1 - protein, 5 aa, Artificial Sequence
  Synthetic
ELGLG

SEQ ID NO: 2 - protein, 4 aa, Artificial Sequence
  Synthetic
RGDC

SEQ ID NO: 3 - protein, 5 aa, Artificial Sequence
  Synthetic
GRGDS

SEQ ID NO: 4 - protein, 6 aa, Artificial Sequence
  Synthetic
GRGDSP

SEQ ID NO: 5 - protein, 5 aa, Artificial Sequence
  Synthetic
RGDYK

SEQ ID NO: 6 - protein, 11 aa, Artificial Sequence
  Synthetic DV3 Peptide
LGASWHRPDKC

SEQ ID NO: 7 - protein, 9 aa, Artificial Sequence
  Synthetic LYP peptide
CGNKRTRGC

SEQ ID NO: 8 - protein, 14 aa, Artificial Sequence
  Synthetic membrane binding domain of IGFBP3
QCRPSKGRKRGFCW

(19) United States
(12) Patent Application Publication
(10) Pub. No.: US 2017/0136087 A1
(43) Pub. Date: May 18, 2017

(54) MULTIFUNCTIONAL BIODEGRADABLE PEG NANOCARRIER-BASED HYDROGELS FOR PREVENTING HIV TRANSMISSION

(71) Applicant: Rutgers, The State University of New Jersey, New Brunswick, NJ (US)

(72) Inventors: Patrick J. Sinko, Lebanon, NJ (US); Michael L. Chikindas, Highland Park, NJ (US); Stanley Stein, East Brunswick, NJ (US); Yashveer Singh, Highland Park, NJ (US); Sujata Sundara Rajan, Piscataway, NJ (US); Dayuan Gao, East Brunswick, NJ (US); Katia Noll, Mahwah, NJ (US)

(21) Appl. No.: 15/355,618

(22) Filed: Nov. 18, 2016

Related U.S. Application Data
(63) Continuation of application No. 13/885,681, filed on Aug. 20, 2013, now abandoned.
Application No. 13/885,681 is a continuation-in-part of application No. 12/936,392, filed on Dec. 22, 2010, now Pat. No. 9,517,201.
(60) Provisional application No. 61/413,652, filed on Nov. 15, 2010.
(60) Provisional application No. 61/436,320, filed on Jan. 26, 2011.
(60) Provisional application No. 61/042,577, filed on Apr. 4, 2008.

(51) Int. Cl. A61K 38/16; A61K 9/08; A61K 45/06; A61K 9/00

(52) U.S. Cl. CPC A61K 38/164; A61K 9/0034; A61K 9/08; A61K 47/48784; A61K 47/48215; A61K 45/06

(57) ABSTRACT

A multifunctional polyethylene glycol-based hydrogel that includes a multi-arm polyethylene glycol cross-linking unit covalently bound to at least four multi-arm polyethylene glycol nanocarrier units, wherein each nanocarrier unit includes an agent coupled to the nanocarrier unit and each agent is selected from pH-lowering agents, bioadhesion agents, microbicidal-spermicidal agents, and agents that inhibit free and cell-associated HIV binding, provided that each nanocarrier unit comprises a different agent.

### CROSS-REFERENCE TO RELATED APPLICATIONS

This application is a Continuation of U.S. patent application Ser. No. 13/885,681, having a 371(c) date of Aug. 20, 2013, which is the U.S. National Stage of International Patent Application Serial No. PCT/US11/60880, filed Nov. 15, 2011, which claims priority under 35 U.S.C. §119(e) to U.S. Provisional Application Ser. No. 61/413,652, filed Nov. 15, 2010 and U.S. Provisional Application Ser. No. 61/436,320, filed Jan. 26, 2012. This Application is also a Continuation-in-Part of U.S. patent application Ser. No. 12/936,392, having a 371(c) date of Dec. 22, 2010, which is the U.S. National Stage of International Patent Application Serial No. PCT/US09/02164, filed Apr. 6, 2009, which claims priority under 35 U.S.C. §119(e) to U.S. Provisional Application Ser. No. 61/042,577, filed Apr. 4, 2008. The disclosures of the foregoing applications are incorporated herein by reference.

### STATEMENT REGARDING FEDERALLY SPONSORED RESEARCH

This invention was made with government support under Contract No. R01A1084137-01 awarded by the National Institutes of Health HIT-IT program. This invention was also made with government support under Contracts No. NCCAM NIH R21AT002897-01, NIH AR055073, NIH AI084137, and NIH AT002897, all of which were awarded by the National Institutes of Health. The government has certain rights in the invention.

### BACKGROUND OF THE INVENTION

Bacterial vaginosis (BV) is a common condition characterized by an imbalance in the vaginal microflora, where healthy lactobacilli are replaced by a proliferation of facultative and anaerobic microorganisms, most notably Gardnerella vaginalis and Prevotella, Peptostreptococcus, Porphyromonas, and Mobiluncus spp. BV-associated microorganisms can be considered STD-causing agents. While some researchers and medical doctors do not consider BV a sexually transmitted disease, several groups have reported on the sexual transmission of BV.

It has been estimated that between 10-30% of women in North America are afflicted by this ailment, frequently prompting them to seek medical attention. Although BV often remains asymptomatic, the unrestricted growth of these organisms has been demonstrated to have pathogenic effects, particularly in pregnant women. BV is associated with the development of pelvic inflammatory disease, as well as a variety of pregnancy-related complications, including low fetal birth weight, preterm births with an elevated risk of infant death, intra-amniotic infections leading to fetal brain damage, and spontaneous abortion. Additionally, Bacterial vaginosis, and G. vaginalis in particular, has been shown to increase the probability of contracting HIV and to stimulate its proliferation in multiple cell lines.

With the incidence of HIV infection on the rise, the development of vaccines and topical microbicides has been a major worldwide priority. However, the results of recent trials have been disappointing. As such, the induction of sterilizing immunity and protection against HIV infection continues to be a major public health goal. “Microbicides,” topically applied agents that prevent HIV transmission from person to person, are still believed to hold considerable promise. In fact, it has been estimated that a microbicide used 50% of the time by 20% of women at risk could prevent 2.5 million HIV infections in 3 years. Given recent clinical developments, there is an urgent need to rethink the concept of microbicides.

### SUMMARY OF THE INVENTION

The present invention is directed to a multifunctional polyethylene glycol-based hydrogel that includes a multi-arm polyethylene glycol cross-linking unit covalently bound to at least four multi-arm polyethylene glycol nanocarrier units, wherein each nanocarrier unit includes an agent coupled to the nanocarrier unit and each agent is selected from pH-lowering agents, bioadhesion agents, microbicidal-spermicidal agents, and agents that inhibit free and cell-associated HIV binding, provided that each nanocarrier unit comprises a different agent. at least two nanocarrier units comprise an agent having a different functionality

In one embodiment, at least one agent is coupled to a nanocarrier unit via a degradable bond. In another embodiment, at least one agent is coupled to a nanocarrier via a nondegradable bond. In yet another embodiment, the hydrogel includes a pH-lowering agent selected from lactic acid, citric acid, ascorbic acid, and maleic acid.

In one embodiment, the hydrogel includes a pH-lowering agent encapsulated in a carrier. In another embodiment, the carrier is cyclodextrin, a dendron, a dendrimer, a liposome, or a PEG nanogel particle.

In yet another embodiment, the hydrogel includes subtilosin. In yet another embodiment, the hydrogel includes an agent that inhibits free and cell-associated HIV binding selected from soluble polyanions and an RGD peptide ligand. In another embodiment, the soluble polyanion is selected from dextran sulfate, cyclodextrin sulfate, and heparin. In yet another embodiment, the hydrogel includes at least one nanocarrier unit noncovalently bound within the hydrogel.

The present invention also relates to a method for preparing a hydrogel by combining an amount of multi-arm polyethylene glycol cross-linking units that include a thiol-reactive functional group coupled to each arm with an amount of multi-arm polyethylene glycol nanocarrier units, wherein each nanocarrier unit includes a thiol group coupled to half of the arms and an agent coupled to the remaining arms of each nanocarrier unit and each agent is selected from pH-lowering agents, bioadhesion agents, microbicidal-spermicidal agents, and agents that inhibit free and cell-associated HIV binding; wherein said amounts of the cross-linking units and the nanocarrier units are sufficient to produce a hydrogel when combined. In one embodiment, each nanocarrier unit that is combined with the same polymer unit includes a different agent.

Also presented is a kit for use in preparing a multifunctional polyalkylene oxide-based hydrogel that includes: (a) an amount of multi-arm polyethylene glycol cross-linking units that include a thiol-reactive functional group coupled to each arm; and (b) an amount of multi-arm polyethylene glycol nanocarrier units, wherein each nanocarrier unit includes a thiol group coupled to half of the arms and an agent coupled to the remaining arms of each nanocarrier unit and each agent is selected from pH-lowering agents, bioadhesion agents, microbicidal-spermicidal agents, and agents that inhibit free and cell-associated HIV binding; wherein the amounts of the cross-linking units and the nanocarrier units are sufficient to produce a hydrogel when combined.

Also provided is a method for prophylactically reducing the risk of development of HIV in a patient by intravaginally or intrarectally administering to a patient: (a) an amount of multi-arm polyethylene glycol cross-linking units that include a thiol-reactive functional group coupled to each arm; and (b) an amount of multi-arm polyethylene glycol nanocarrier units, wherein each nanocarrier unit includes a thiol group coupled to half of the arms and an agent coupled to the remaining arms of each nanocarrier unit and each agent is selected from pH-lowering agents, bioadhesion agents, microbicidal-spermicidal agents, and agents that inhibit free and cell-associated HIV binding; wherein the amounts of the cross-linking units and the nanocarrier units are sufficient to produce a hydrogel when combined.

Also provided is an article that includes the hydrogel of the present invention.

Another embodiment includes a topical composition that includes an anti-microbial and/or spermicidal effective amount of subtilosin incorporated into a pharmaceutically acceptable aqueous solution, non-aqueous solution, nanofiber, hydrogel, gel, nanogel, suspension, ointment, jelly, insert, suppository, sponge, salve, cream, foam, foaming tablet, or douche.

### BRIEF DESCRIPTION OF THE DRAWINGS

FIG. 1 are photographs of (left panel) syringes with separate solutions of crosslinker and polymer and (Right Panel) the hydrogel formed from crosslinker and polymer. Blue dye was added to the polymer solution in order to visualize the hydrogel otherwise it is clear and colorless;

FIG. 2 is a reaction scheme showing the SH on the polymer (left) reacts with the TP group on the crosslinking nanocarrier (right) to form hydrogel. When the gel degrades, the nanocarrier is released;

FIG. 3 depicts TEM images of 5% hydrogels from copolymer and crosslinker in 1:1 stoichiometry. The crosslinking networks are clearly visible;

FIG. 4 is a graph showing the influence of strain (G′ and G″) on 3% and 5% (w/v) hydrogels prepared from copolymer and crosslinker with 1:1 stoichiometry. The frequency sweep test indicates that the hydrogels are highly elastic and that they have the ability to resist structural changes under strain that occurs during physical activity;

FIG. 5A depicts a synthetic scheme for attaching RGD peptides to a PEG nanocarrier;

FIG. 5B depicts another synthetic scheme for attaching RGD peptides to a PEG nanocarrier;

FIG. 6 are DSC results showing (a) the Tₘ for 8 arm PEG-SH is 53.3° C., (b) The Tₘ for intermediate 8 arm PEG-S-TP is 45.81° C. (shift of −7.56° C.), and (c) Tₘ for RGD linked to PEG nanocarrier is 37.69° C. (The shift of −15.6° C. confirms the conjugation);

FIG. 7 is a reaction scheme depicting the synthesis of PEG-LA nanocarriers; (a) dichloromethane (DCM), dicyclohexylcarbodiimide (DCC), RT, 8 h; and (b) DCM, dimethylaminopyridine (DMAP), RT, 4 h;

FIG. 8A is shows the GPC profiles of PEG_{20 kDa}-LA (4-arm);

FIG. 8B is shows the GPC profiles of PEG_{20 kDa}-LA (8-arm);

FIG. 9A are plots showing cumulative release of lactic acid from 8-arm PEG-LA nanocarriers in PBS (pH 7.4) and acetate (pH 4.3) buffers; Mean±S.E., n=3. The release profile was fitted using a one-phase exponential association equation. The bottom panel shows the release profile for first 12 h;

FIG. 9B shows the cumulative release of lactic acid from nanocarrier-based hydrogels in (A) PBS (pH 7.4) and (B) acetate buffer (pH 4.3); Mean±S.E., n=3. The data were fit with a one-phase exponential association equation (A) or two-phase exponential association equation (B) ^{a}n=2;

FIG. 9C shows the cumulative release of lactic acid in PBS (pH 7.4) from hydrogels containing passively entrapped lactic acid; Mean±S.E., n=3. The release profile was fit using a one-phase exponential association equation. ^{a}n=2 and ^{b}n=1.

FIG. 10 is a schematic representation of hydrogel formation using 8-arm PEG_{20 kda}-LA nanocarrier and 4-arm PEG20 kDa-NHS crosslinker;

FIG. 11 is a table providing the time of formation of PEG-LA nanocarrier-based hydrogels;

FIG. 12 is a plot showing swelling and degradation of PEG-LA nanocarrier-based hydrogels in PBS (pH 7.4) and acetate buffer (pH 4.3); Mean±S.E., n=3.

FIG. 13 is a synthetic scheme for producing PEG nanogels (left) and a representative PEG nanogel aggregate (right);

FIG. 14 are TEMs of PEG nanogels (Panel A), micron-sized stable nanogel aggregates (Panel B) and large (>100 micron) PEG nanogel aggregates;

FIG. 15 is a plot showing EpiVaginal tissue viability following exposure to subtilosin A. The bars are the average of two independent experiments;

FIG. 16 is a plot showing subtilosin A immobilizes human spermatozoa in a dose-dependent manner. The percentage of motile spermatozoa in pooled whole semen was determined 30 seconds after mixing with subtilosin A, at different final concentrations, as indicated. All data were adjusted to a normal control motility of 70% and subjected to arcsine transformation before further analysis. Values are expressed as average % motility. Error bars are 90% confidence limits;

FIG. 17A is a synthetic scheme for crosslinking nanocarriers. (Left Panel): Scheme for attaching subtilosin. (Right Panel): Scheme for synthesizing polyanionic nanocarriers. The same procedure (n=1) will be used for the pH lowering nanocarriers;

FIG. 17B is another scheme for attaching subtilosin to a nanocarrier;

FIG. 18 is a conceptual drawing of the formation of a multiplex hydrogel from the various crosslinking nanocarriers. In addition, particulates can be passively entrapped in the gel matrix.

FIG. 19 is a table setting forth the growth conditions and subtilosin sensitivity of indicator organisms;

FIG. 20 is a table providing the effect of enzymatic digestion on antimicrobial activity;

FIG. 21 is a table listing the specific primers for the functional genes of subtilin and subtilosin;

FIG. 22 is a table providing the concentrations of D- and L-lactic acid in CFS;

FIG. 23 is a table providing the effect of temperature stress on antimicrobial activity;

FIGS. 24A and 24B are graphs comparing ATP levels;

FIGS. 25A and 25B are plots demonstrating that subtilosin has no effect on transmembrane electric potential (ΔΨ) in G. vaginalis cells;

FIGS. 26A and 26B are plots demonstrating that subtilosin depletes the transmembrane pH gradient (ΔpH) in G. vaginalis cells;

FIG. 27 is a table setting forth ectocervical cell viability after prolonged exposure to subtilosin;

FIG. 28 is a table setting forth the minimal inhibitory concentrations (MICs) of subtilosin, glycerol monolaurate, lauric arginate, poly-lysine, and zinc lactate against the BV-associated pathogen G. vaginalis;

FIG. 29 is an isobologram showing the individual MICs for glycerol monolaurate (GML) (20 μg/mL) and subtilosin (9.2 μg/mL) connected by a trendline;

FIG. 30 is a table providing the minimal inhibitory concentrations (MICs) of antimicrobial compounds tested in a checkerboard assay against G. vaginalis;

FIG. 31 is an isobologram showing the individual MICs for lauric arginate (LAE) (100 μg/mL) and subtilosin (9.2 μg/mL) connected by a trendline;

FIG. 32 is an isobologram showing the individual MICs for subtilosin (9.2 μg/mL) and poly-lysine (25 μg/mL) connected by a trendline; and

FIG. 33 is an isobologram showing the individual MICs for zinc lactate (1090.1 μg/mL) and subtilosin (9.2 μg/mL) connected by a trendline.

FIG. 34 is a graph showing the influence of strain (G′ and G″) on 4% and 6% (w/v) PEG-LA nanocarrier-based hydrogels as a function of (A) Frequency and (B) Strain.

### DETAILED DESCRIPTION OF THE INVENTION

The present invention relates to a multiplex nanocarrier-based polyethylene glycol (PEG) vaginal hydrogel for preventing the initial infection (i.e., acquisition) and dissemination of HIV through the vaginal mucosa to distant tissues. The multiplex hydrogel matrix is formed by crosslinking various PEG nanocarriers, each of which plays a different role in the functional properties of the hydrogel (e.g., promoting mucosal adhesion, maintaining mildly acidic pH, releasing microbicide and spermicides, and preventing HIV virion binding).

Hydrogels are formed by intermolecular cross-linking of hydrophilic polymers. They are capable of absorbing large amounts of water and swelling, while maintaining their three-dimensional networks. Molecules of different sizes can diffuse through the hydrogel matrix, which resembles living tissue due to the hydrogel's high-water content and soft/rubbery characteristics. Hydrogels are used in drug delivery, tissue engineering, and imaging applications. The current polymer and crosslinker nanocarriers are based on polyethylene glycol, which is a water soluble, nontoxic and biocompatible polymer. This is particularly important since disruption of the mucosal tissues and normal vaginal flora has been associated with increased rates of HIV-1 acquisition and shedding. The hydrogel is a liquid upon instillation allowing for high vaginal dispersion and mucosal coverage where it then undergoes a rapid phase transition to form a visco-elastic hydrogel that does not depend upon temperature or pH. The multiplex hydrogel matrix is formed by crosslinking various PEG nanocarriers each of which plays a different role in the functional properties of the hydrogel (e.g., promoting mucosal adhesion, maintaining mildly acidic pH, releasing microbicides and spermicides, and preventing HIV virion binding).

It has been shown that (1) sexually transmitted and vaginal infections such as bacterial vaginosis (BV) increase the risk of HIV transmission by weakening mucosal barriers and by stimulating an inflammatory response that may activate or recruit HIV target cells to the portals of viral entry, (2) low vaginal pH (<4.5) inactivates HIV and inhibits CD4+ lymphocyte activation, thus reducing the number of HIV target cells in the vagina, and (3) cell-associated HIV breaches the normal stratified squamous epithelial barrier of the vagina with low frequency.

Preferably, the hydrogel of the present invention imparts a robust physical barrier, restores the natural microbicidal vaginal barrier functionality, and prevents HIV binding. It is also preferred that the hydrogel is colorless, odorless, inexpensive to manufacture, safe to use more than once a day and for long periods of time, fast-acting, undetectable to either partner, and available in contraceptive and noncontraceptive forms. For example, the hydrogel can be applied directly to the vagina or rectum.

The hydrogel of the invention may also be impregnated into absorptive substrate materials, such as sponges, or coated onto the surface of solid substrate materials, such as male or female condoms, diaphragms, cervical caps, or medical gloves, to deliver the compositions to vaginal or other potentially infectable epithelium. Other articles and delivery systems of this type will be readily apparent to those skilled in the art.

As used herein, “condom” refers to a barrier device which is used to provide a watertight physical barrier between male and female genitalia during sexual intercourse, and which is removed after intercourse. This term includes conventional condoms, which cover the penis; it also includes so-called “female condoms” which are inserted into the vaginal cavity prior to intercourse. Preferably, condoms should be made of latex or a synthetic plastic material such as polyurethane, since these provide a high degree of protection against viruses.

Also provided is a method for prophylactically reducing the risk of development of HIV in a patient by intravaginally or intrarectally administering to a patient: (a) an amount of multi-arm polyethylene glycol cross-linking units that include a thiol-reactive functional group coupled to each arm; and (b) an amount of multi-arm polyethylene glycol nanocarrier units, wherein each nanocarrier unit includes a thiol group coupled to half of the arms and an agent coupled to the remaining arms of each nanocarrier unit and each agent is selected from pH-lowering agents, bioadhesion agents, microbicidal-spermicidal agents, and agents that inhibit free and cell-associated HIV binding; wherein the amounts of the cross-linking units and the nanocarrier units are sufficient to produce a hydrogel when combined.

In one embodiment of the present invention, an 8-arm PEG polymeric nanocarrier is crosslinked to form the hydrogel network entrapping water as the hydrogel forms. As a result, hydrogels resemble living tissue due to their high-water content and soft/rubbery characteristics. The PEG hydrogel can serve as a lubricant during sex. The basic PEG unit is identical for the “cross-linking” and “nanocarrier” units with two exceptions: (1) functional groups on the cross-linking unit (e.g. thiol-reactive functional groups, including but not limited to activated ester, activated thiol, maleimide, vinyl sulfone, and the like) and on the nanocarrier unit (e.g. thiol (—SH)) are complimentary so that they will react to form the hydrogel network and (2) in addition to the thiol-reactive functional groups, the nanocarrier units also possess various functionalities such as pH lowering units, bioadhesion units (e.g. xanthan gums, hydroxypropyl cellulose, carpools, polycarbophils, chitosan, alginates, and the like), microbicide & spermicide units or polyanionic and RGD units to block free and cell-associated HIV binding (e.g. antiviral). Mixing various nanocarrier units with the cross-linking unit forms the multiplex hydrogel, imparting the desired functional properties to the hydrogel.

The nanocarrier units are prepared using multi-arm and/or branched PEG-thiol polymers. The number of thiol groups (e.g. arms) varies preferably from 2 to 16, more preferably from 2 to 8. The molecular weight of the thiol polymer ranges preferably from about 10,000 Da to about 100,000 Da, more preferably from about 10,000 Da to about 60,000 Da. The amount of nanocarrier units used to prepare the hydrogels of the present invention varies from about 2% w/v to about 40% w/v, more preferably from about 2% w/v to about 20% w/v. In one embodiment of the present invention, the hydrogel includes copolymers containing repeating units of thiol groups, e.g. poly[poly(ethylene glycol)-alt-poly(mercaptosuccinic acid)] having a molecular weight range from about 1,000 Da to about 100,000 Da.

The cross-linking unit is either linear or a multi-arm (branched) polymer that includes thiol-reactive functional groups, such as, activated esters, activated thiols, maleimide, vinyl sulfone, and the like. The molecular weight range for the cross-linking unit preferably ranges from about 1,000 Da to about 40,000 Da, more preferably from about 2,000 Da to about 20,000 Da. The number of functional groups varies preferably from 2 to 8. The nanocarrier unit to cross-linking unit stoichiometry varies from 10:0.05 to 0.05:10.

Currently marketed vaginal gels (e.g. Conceptrol II® and Gynol II®) are “soft” gels that use gelling agents such as sodium carboxymethylcellulose to increase their viscosity. As a result, soft gels have poor mechanical strength and are unable to maintain a robust physical barrier to pathogens. It is equally important that vaginal gels have good viscoelastic properties in order to resist structural changes under strain (e.g., during normal movement, sexual intercourse, etc.). If a gel cannot resist structural changes, openings will form in the gel allowing pathogens to invade the mucosa. To our knowledge, none of the gels that are currently marketed or are being developed have any significant elastic nature. It is also imperative that gels have high disperability and retention inside the vagina to insure maximal mucosal surface coverage. Physical gels have limited ability to spread and cover the mucosal surface once instilled into the vagina (i.e., spreading only occurs as the gel becomes diluted and less viscous making it an even less effective barrier). It is readily apparent that currently marketed vaginal gels were not designed to provide a good physical barrier to pathogens. The hydrogel of the present invention offers the advantage of being administered as a solution in order to get maximal vaginal mucosal coverage. However, unlike any of the commercially available gels, it then quickly forms a firm hydrogel of good viscosity, flexibility/elasticity and mechanical strength.

The functional properties of the hydrogel of the present invention are customized by covalently linking an agent with a nanocarrier unit of the hydrogel or by passively (i.e., noncovalently) trapping it within the hydrogel matrix as it forms in situ. A higher loading capacity of an agent can be achieved by passive entrapment, however, high agent payloads may not always be needed. For example, if the goal is to maintain vaginal pH or slightly reduce pH then the covalently linked acids should be adequate because their release will be slow and sustained. If the goal is to dramatically reduce pH (e.g., during the initial treatment of BV) then higher “doses” with a shorter duration of release will be required. Passive entrapment can also be used to achieve this functionality In one embodiment, one or more agents are functionalized with thiol-reactive functional groups, which include but are not limited to activated esters, activated thiols, vinyl sulfone, malemide, and the like to form either degradable thioester and disulfide bonds or stable (non-degradable) thioether bonds with the polymer. The number of agents attached to the polymer varies from preferably 1 to 8, more preferably from 1 to 4. In terms of amount, the agents account for thiol modification in the range of from about 10% to about 80%, more preferably from about 10% to about 60%. In one embodiment, noncleavable linkages are used for the HIV binding functionality whereas cleavable linkages are used for releasing therapeutic agents.

Preferably, the hydrogel of the present invention restores a normal microbicidal vaginal environment and thus prevents HIV transmission by effectively maintaining acidic pH and treating BV infection. Vaginal infections such as BV and the introduction of semen, which is alkaline, into the vagina elevate pH above the critical pH (˜4.5) required to inactivate HIV and BV pathogens. The altered vaginal environment is favorable to HIV entry and transmission. Unfortunately, most attempts at maintaining acidic vaginal pH have failed due to poor delivery methods of the acidifying agent and/or low buffer capacity. The hydrogel of the present invention mimics the function of the natural vaginal environment by slowly releasing low amounts of lactic acid or other safe mild acids.

Lactic acid is the preferred acidifying agent due to its natural function in the vagina. Lactic acid-nanocarriers are formed by reacting the N-hydroxysuccinimidyl ester of lactic acid with the —SH groups of the polymer via a thioester linkage. The thioester linkages degrade, slowly releasing lactic acid. At the pH of the diseased vagina (pH 5-7), lactic acid is preferably released over a period of 18-30 hours. Lactic acid is attached either directly or through a linker, which preferably 2-12, and more preferably 2-6 carbons long. The number of lactic acid moieties on the polymer varies preferably from 1 to 8, more preferably from 1 to 4. Citric, maleic, or ascorbic acid can also be used. The linker for citric acid is mercaptoethanol and 3-mercaptopropanoic acid for ascorbic acid. An alternative preparation is to encapsulate the acids in a carrier such as cyclodextrin, dendrons, dendrimers, liposomes, or PEG nanogel particles, such as those disclosed in International Publication No. WO2009123768, the contents of which are incorporated herein by reference, and passively entrap those particles in the hydrogel where they slowly release the acids.

In another embodiment, the hydrogels of the present invention treat bacterial vaginosis (BV). B. subtilis produces a lesser-known bacteriocin, subtilosin A, a circular peptide of 35 amino acids, with the distinctive post-translational modification of three sulfur cross-links between cysteine and the alpha-carbon of two phenylalanines and one threonine residue. This peptide, unique among bacteriocins, is pharmacologically active against BV pathogens and is spermicidal. In one embodiment, subtilosin is incorporated into the hydrogel for use as a natural microbicidal-spermicidal agent to treat BV. The free carboxyl group on glutamic acid present at position 23 is activated and reacted with thiol groups on the polymer to form degradable thioester bonds. Alternatively, the subtilosin is attached using the amine functional group on lysine moiety. The subtilosin is attached to the polymer either directly or through a linker, which is preferably 2-12, and more preferably 2-6 carbons long. The number of subtilosin moieties on the polymer varies preferably from 1-8, more preferably from 1-4.

In yet another embodiment, the hydrogels of the present invention prevent HIV binding to cells to reduce HIV transmission. Nonspecific attachment inhibitors can be active against both free- and cell-associated HIV. The first step of HIV binding involves the interaction with a target cell. This nonspecific adsorption/attachment process, which occurs before gp120 binding to CD4, is based on the interaction of the positively charged regions of Env with the negatively charged proteoglycans of the cell surface. Soluble polyanions, such as dextran sulfate, cyclodextrin sulfate, and heparin, have been shown to block the nonspecific attachment of HIV virions. A number of other polyanions have also been reported to have such activities as well.

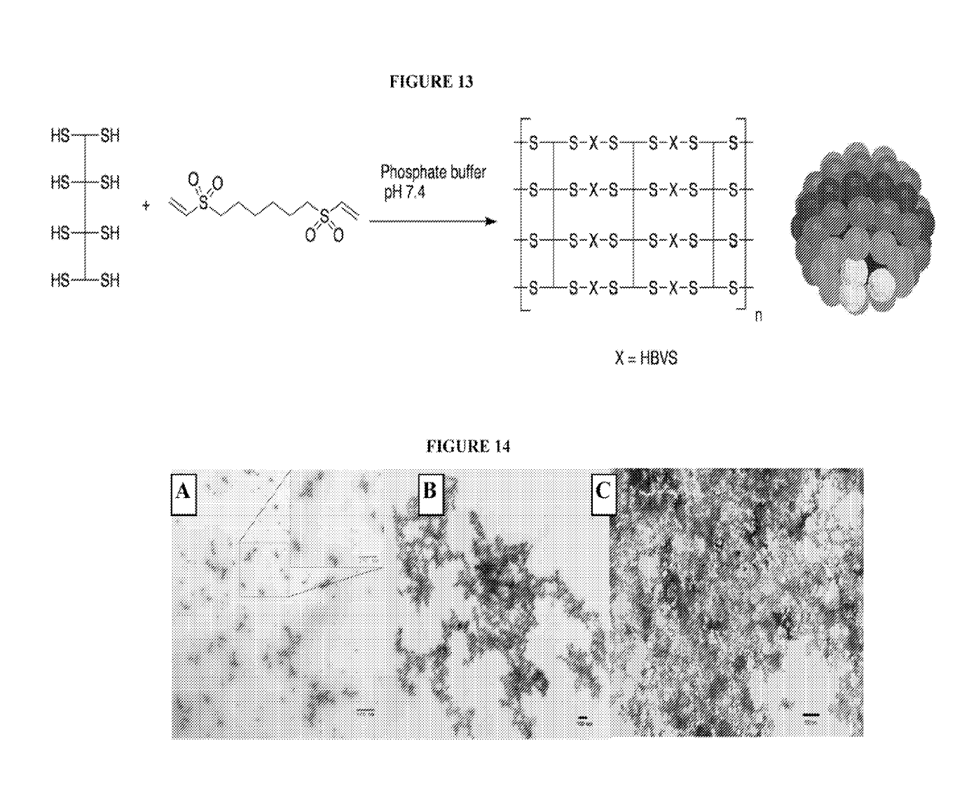

In another embodiment of the present invention, polyanionic nanocarriers are constructed by attaching negatively charged amino acids, which include but are not limited to Glu and Asp. These amino acids have two carboxylic groups (two negative charges). The amino acids are attached to the polymer either directly or through a linker, which is preferably 2-12, and more preferably 2-6 carbons long. The anionic amino acids are attached to the polymer preferably through non-degradable bonds. The number of amino acids on eight-arm thiol polymers varies from 1-8, more preferably from 1-4. Since each amino acid has two anionic charges, the negative charge on the nanocarrier ranges from 2-16, more preferably from 2-8. In another embodiment, charge density is increase 2-3 fold by using di- or tripeptide instead of amino acids. Other examples of polyanions include, but are not limited to, dextran sulphate, heparin sulfate, and the like. Another embodiment utilizes aggregated PEG nanogels (FIGS. 13 and 14). These micron-sized particles are similarly functionalized and passively entrapped in rather than covalently linked to the hydrogel.

Another binding interaction is based on the interaction of the peptide ligand RGD with αβ (e.g. αᵥβ₃, α₅β₁, etc.) integrins on cell surfaces. It has been suggested that HIV-1 entry into the vaginal mucosal epithelial cells is more efficient when HIV-1 particles bud locally after contact between HIV-1-infected cells and uninfected mucosal epithelial cells rather than by direct entry of cell-free virus into the epithelial cells. This interaction, also true for CD4⁺ T-cells, is integrin and proeteoglycan agrin-dependent. In one embodiment of the present invention, the RGD-peptide is attached to the thiol polymer through non-degradable bonds. The RGD peptide is either linear or cyclic and is attached either directly or through linker, which is preferably 2-12, and more preferably 2-6 carbons long. The number of RGD peptide on the polymer varies, preferably from 1-8, and more preferably from 1-4.

Preferably, the hydrogels in present invention also treat the HIV infection. The preferred therapeutic is nucleotide reverse transcriptase inhibitor (NRTI), tenofovir. Tenofovir is an analogue of adenosine monophosphate, and is characterized as acyclic nucleoside phosphate. Tenofovir is administered orally as prodrug, tenofovir disoproxil fumarate. It is converted to its active form, tenofovir diphosphate, intracellularly by phosphorylation, and acts as a chain terminator when HIV reverse transcriptase is actively making viral DNA.

In one embodiment of the present invention, tenofovir is attached to the thiol polymer via degradable thioester bonds. Tenofovir is attached either directly or through linker, which is preferably 2-12, and more preferably 2-6 carbons long. The tenofovir moieties on the polymer vary preferably from 1-8, more preferably from 1-4. In another embodiment of present invention, a therapeutic agent is passively encapsulated into the hydrogel matrix. Other examples of therapeutic agents include, but not limited to, UC781; nucleoside reverse transcriptase inhibitors (NRTIs) like zidovudine, didanosine, zalcitabine, stavudine, lamivudine, abacavir sulfate, emtricitabine, etc.; non-nucleoside reverse transcriptase inhibitors (NNRTIs) like nevirapine, delaviridine, efavirenz, entavirine, etc.; protease inhibitors (PIs) like saquinavir mesylate, ritonavir, indinavir, nelfinavir mesylate, amprenavir, fosamprenavir calcium, atazanavir sulfate, lopinavir and ritonavir, tipranavir, darunavir, etc.; entry and fusion inhibitors like maraviroc, enfuviritide, etc.; and integrase inhibitors like raltegravir etc.

Methods for preparing the hydrogels of the present invention are also presented. In one embodiment, the hydrogel is prepared by combining an amount of multi-arm polyethylene glycol cross-linking units that include a thiol-reactive functional group coupled to each arm with an amount of multi-arm polyethylene glycol nanocarrier units, wherein each nanocarrier unit includes a thiol group coupled to half of the arms and an agent coupled to the remaining arms of each nanocarrier unit and each agent is selected from pH-lowering agents, bioadhesion agents, microbicidal-spermicidal agents, and agents that inhibit free and cell-associated HIV binding; wherein said amounts of the cross-linking units and the nanocarrier units are sufficient to produce a hydrogel when combined. In one embodiment, each nanocarrier unit that is combined with the same polymer unit includes a different agent.

Also presented is a kit for use in preparing a multifunctional polyalkylene oxide-based hydrogel that includes: (a) an amount of multi-arm polyethylene glycol cross-linking units that include a thiol-reactive functional group coupled to each arm; and (b) an amount of multi-arm polyethylene glycol nanocarrier units, wherein each nanocarrier unit includes a thiol group coupled to half of the arms and an agent coupled to the remaining arms of each nanocarrier unit and each agent is selected from pH-lowering agents, bioadhesion agents, microbicidal-spermicidal agents, and agents that inhibit free and cell-associated HIV binding; wherein the amounts of the cross-linking units and the nanocarrier units are sufficient to produce a hydrogel when combined.

The present invention also relates to methods for prophylactically reducing the risk of development of bacterial vaginosis in a patient by intravaginally administering a composition to the patient that includes a bacterial vaginosis prophylactic effective amount of subtilosin. In another embodiment, the composition further includes an antimicrobial selected from consisting of glycerol monolaurate, lauric arginate, poly-lysine, and zinc lactate.

Also provided are methods for treating bacterial vaginosis in a patient by intravaginally applying a treatment effective amount of subtilosin to the patient or a treatment effective amount of subtilosin and an antimicrobial selected from glycerol monolaurate, lauric arginate, poly-lysine, and zinc lactate to the patient. The present invention also relates to compositions that include an anti-microbial and/or spermicidal effective amount of subtilosin incorporated into a pharmaceutically acceptable aqueous solution, non-aqueous solution, nanofiber, hydrogel, gel, nanogel, suspension, ointment, jelly, insert, suppository, sponge, salve, cream, foam, foaming tablet, or douche.

Subtilosin A (commonly referred to as subtilosin) is produced by both Bacillus subtilis and Bacillus amyloliquefaciens and has a cyclical, cross-linked structure unique among characterized bacteriocins. Bacteriocins are ribosomally-synthesized peptides produced by bacteria that have antimicrobial activity against organisms closely related to the producer species.

“Bacterial vaginosis prophylactic effective amount” is used herein to mean that amount which results in a sufficient concentration of subtilosin at a desired site to inhibit the development of bacterial vaginosis in a patient.

“Treatment effective amount” is used herein to mean that amount which results in a sufficient concentration of subtilosin at an infected site to therapeutically ameliorate or reduce the effects of the disease. The disease being treated can be the first occurrence or a subsequent reoccurrence of the disease in the patient.

“Anti-microbial effective amount” is used herein to mean that amount which results in a sufficient concentration of subtilosin to kill or inhibit the growth of one or more microorganisms (e.g. facultative and anaerobic microorganisms including but not limited to Gardnerella vaginalis and Prevotella, Peptostreptococcus, Porphyromonas, and Mobiluncus spp.; wild-type bacteria; and antibiotic-resistant bacterial vaginosis-associated bacteria.).

“Spermicidal effective amount” is used herein to mean that amount which results in a sufficient concentration of subtilosin to kill or disable sperm.

The compositions used in the instant invention may be applied topically to prevent or treat bacterial vaginosis or kill or disable sperm. For topical administration, suitable carriers or vehicles include polar, protic solvents, such as, water or normal saline, non-polar solvents, lipids, ointments, jellies, inserts and foaming inserts (suppositories, sponges, and the like) salves, creams, foams, douches, nanofibers, hydrogels, gels, nanogels, or the like. The compositions may also be suspended in a suspension medium that is not miscible with water, for example, petrolatum, or may be formulated in an emulsion (water-in-oil or oil-in-water). More particularly, the compositions can be applied intravaginally for the prevention or treatment of bacterial vaginosis. The topical composition containing subtilosin could, for example, be applied with an applicator or an intravaginal device or the topical composition could be coated on a male or female condom or other sexual barrier devices, such as diaphragms, cervical caps, and the like.

For topical applications, the pharmaceutically acceptable carrier may additionally comprise organic solvents, emulsifiers, gelling agents, moisturizers, stabilizers, surfactants, wetting agents, preservatives, time-release agents, and minor amounts of humectants, sequestering agents, dyes, perfumes, and other components commonly employed in pharmaceutical compositions for topical administration.

Solid dosage forms for topical administration include suppositories, powders, and granules. In solid dosage forms, the compositions may be admixed with at least one inert diluent such as sucrose, lactose, or starch, and may additionally comprise lubricating agents, buffering agents and other components well known to those skilled in the art.

The compositions of the invention may also be impregnated into absorptive substrate materials, such as sponges, or coated onto the surface of solid substrate materials, such as male or female condoms, diaphragms, cervical caps, or medical gloves, to deliver the compositions to vaginal or other potentially infectable epithelium. Other articles and delivery systems of this type will be readily apparent to those skilled in the art.

A method of coating a condom with a composition comprising subtilosin comprises coating the whole surface or necessary portion of a condom by dropping, dipping, coating or spraying a solution containing subtilosin. Condom coating methods are well-known, and the subtilosin compositions can be incorporated into the known condom coating compositions, including lubricant compositions. Preferred coating compositions include silicon, which provides lubricity and releases the composition in a time-release manner In this way, a condom having a spermicidal and/or anti-microbial effect and a lubricating effect can be obtained. Bioadhesive polymers may also be used to prolong the time-release aspects of the particular topical or other medicament employed. Subtilosin can also be impregnated into the condom during manufacture by processes known in the art.

The amount of subtilosin applied on one condom can be any amount that provides the desired prophylactic effect with little or no side effects, preferably from about 0.001 mg to about 1000 mg. Coating a condom is carried out on one side or to both the inner surface and the outer one.

In the present invention, subtilosin is generally administered in such a dosage as to achieve the desired actions with limited or no side effects. Although the actual dosage should be determined according to the judgment of doctors, the preferred concentration in a pharmaceutically acceptable carrier can vary from about 0.00005% to about 5% by weight.

The following non-limiting examples set forth herein below illustrate certain aspects of the invention.

### EXAMPLES

### Characteristics of Nonfunctionalized Hydrogels.

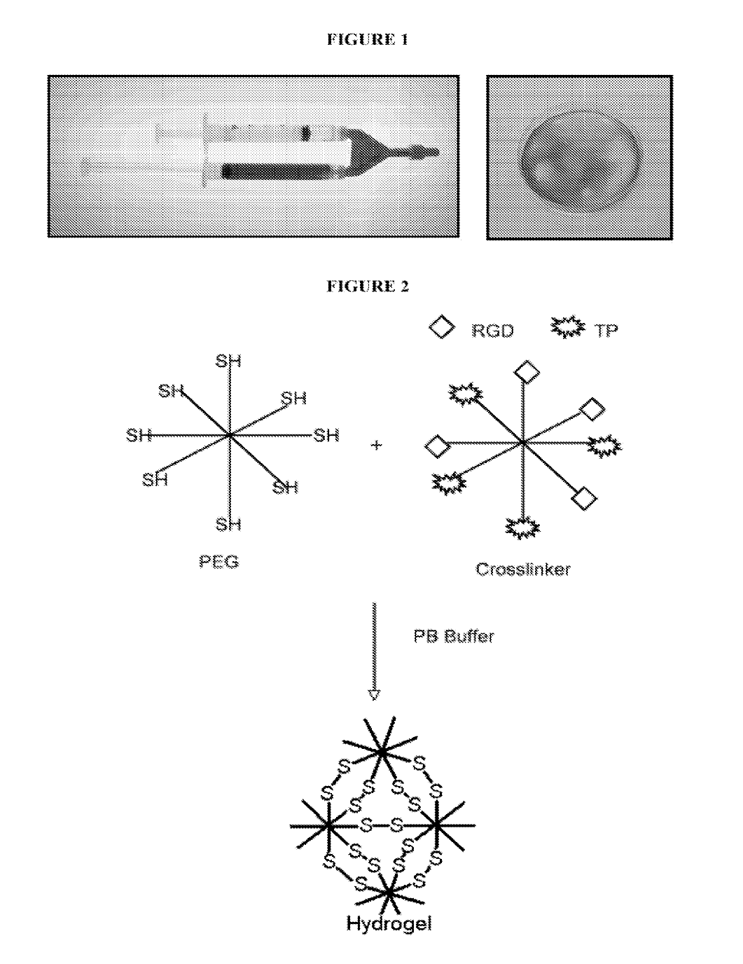

An in situ forming hydrogel has two components, a polymer and a crosslinker. In FIG. 1 (left panel) two solutions are shown, the barrel with the polymer has a blue dye mixed in with it (lower barrel) and the colorless one contains the crosslinker solution (upper barrel). When the plungers are depressed, the liquids mix as they pass through the nozzle and a hydrogel forms instantly (see blue dye-loaded hydrogel on the plate in FIG. 1 right panel). The reaction scheme for producing a hydrogel is shown in FIG. 2. As soon as the polymer and crosslinking nanocarrier meet, a hydrogel network immediate forms even though the solution is free flowing for a period of time. It is possible to passively entrap small particulates in the hydrogel at this time. A firm viscoelastic hydrogel will form. The rate of firming is dependent upon the nature of the crosslinking nanocarrier.

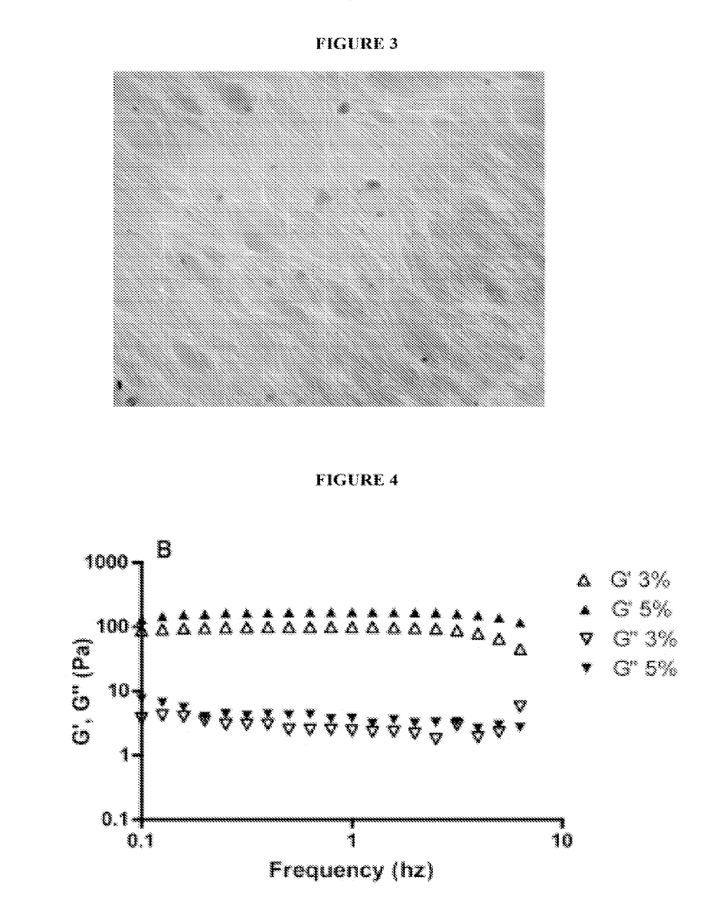

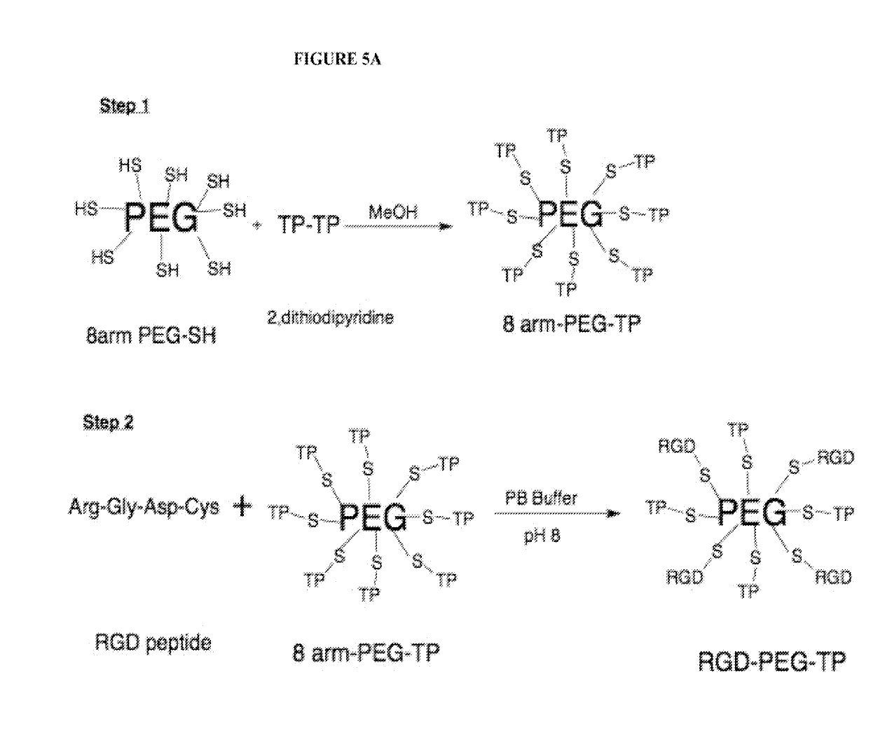

When 50% of the arms have TP groups (as seen in FIG. 2) and 50% are substituted with RGD peptides firm, gellation occurs in about 10 minutes. When all 8 arms have only TP groups, firm gellation occurs in less than one minute. The crosslinking networks are clearly visible in the TEM image of the hydrogel (FIG. 3). A strain sweep test was performed (results not shown) in order to establish the regime of linear viscoelasticity and to examine the differences in elasticity between the two hydrogels with varying amounts of crosslinker (as expressed by the values of storage/elastic modulus, G′). The strain sweep test results show that G′ dominates in both hydrogels and this is supported by the results obtained from the frequency sweep test (FIG. 4) which indicates that the G′ was greater than G″ (loss modulus). This suggests that both hydrogels have high elasticity in the investigated frequency range and should have the ability to resist structural changes under strain. The differences between the two hydrogels are minimal suggesting that varying the polymer and crosslinking components will not severely alter the physical properties of the hydrogel. Based on these results for nonfunctionalized hydrogels, the feasibility of crosslinking nanocarriers to form a hydrogel is demonstrated. It is expected that the hydrogels will possess the physical attributes of a good barrier membrane: they are highly dispersible providing extensive coverage of irregular surfaces; they possess sufficient viscosity to slow viral diffusion; and have outstanding elastic properties to withstand physical strain allowing the hydrogel barrier to remain intact during physical activity.

Adhesive RGD nanocarriers for greater mucosal hydrogel retention and prevention of HIV binding.

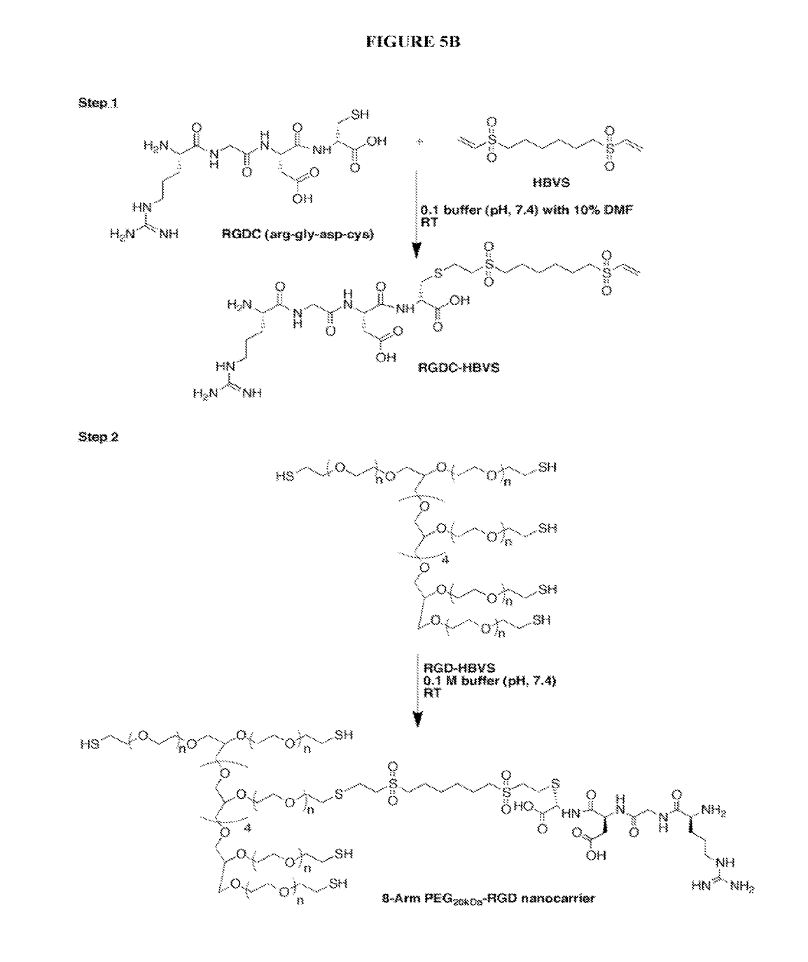

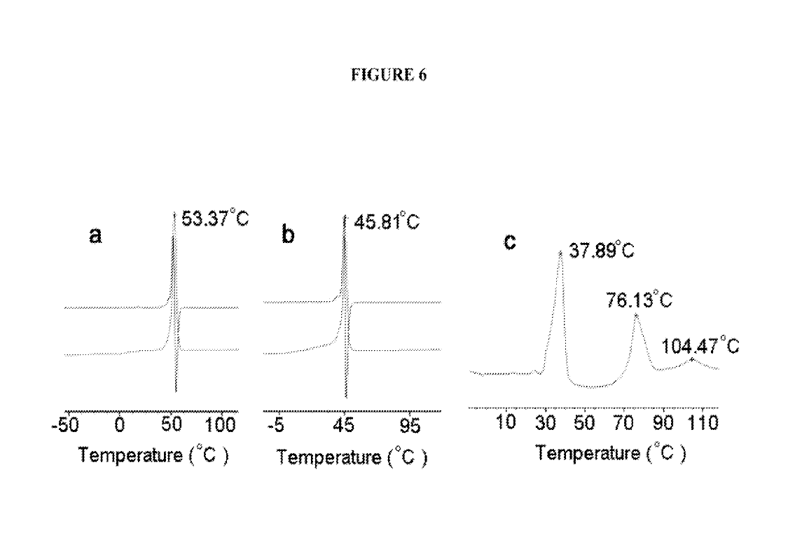

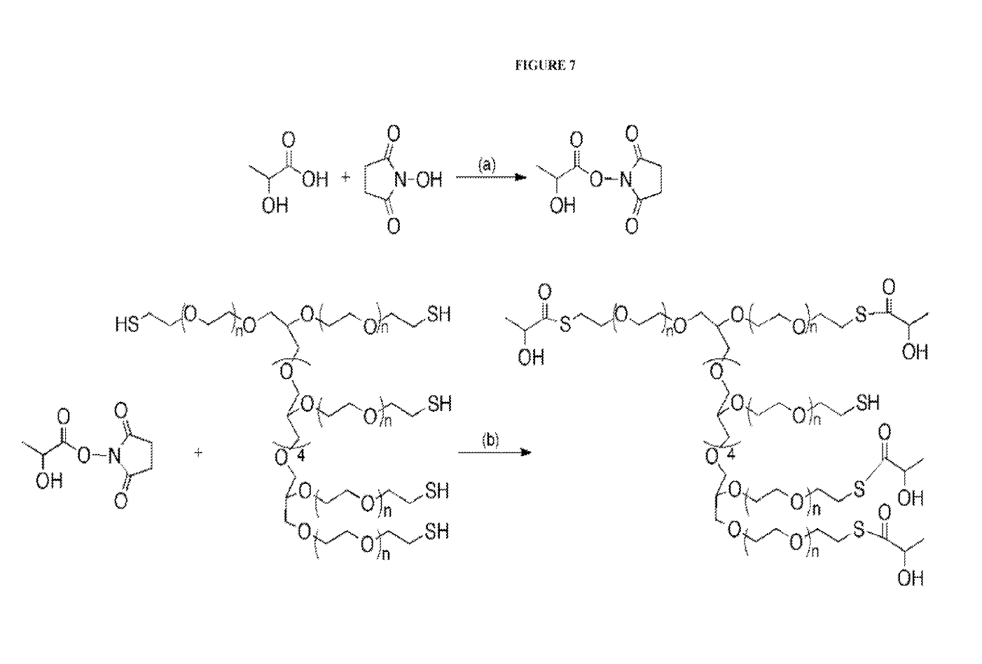

The RGD sequence is known to preferentially bind to α,β-integrins. As a result, RGD adhesive nanocarriers should promote stronger contact between the hydrogel and the vaginal mucosal membrane. The synthesis of a water-soluble RGD-containing nanocarrier is shown. Using the synthetic scheme shown in FIG. 5A, RGD-PEG nanocarriers containing four appended RGD peptides per nanocarrier were synthesized and characterized. ESI-Mass spectrometry, NMR, XPS, and DSC were used to characterize all of the intermediates and final RGD-PEG nanocarrier. DSC results for the 8-arm PEG-RGD nanocarrier are shown in FIG. 6. The shift of −15.6° C. confirms the conjugation of RGD to the PEG. A fluorescent tag (FITC) was attached to the RGD peptide (results not shown) in order to test cell surface adhesion. Synthetic feasibility of preparing the RGD nanocarrier crosslinker is demonstrated.

### Synthesis and Characterization of 4-Arm and 8-Arm PEG-RGD Nanocarriers

RGDC peptide (100 mg, 0.0 mM) was dissolved in sodium phosphate buffer (8 ml, 0.1 M, pH, 7.4) containing 10% dimethylformamide (DMF), and 1,6-hexane-bis-vinyl sulfone (HBVS) (6 equiv., 356.4 mg) was added to it (FIG. 5B, step 1). The reaction mixture was stirred at room temperature for 8 hrs. The product, RGDC-HBVS was purified on silica column using dichloromethane (DCM) as eluent. The conjugate was characterized using MALDI-TOF mass spectrometer.

Four-arm or eight-arm PEG_{20 kDa}-thiol polymer (30 mg) was dissolved in sodium phosphate buffer (0.1 M, pH, 7.4) and the RGDC peptide modified with HBVS linker (3 equiv., 3.2 mg) was added to it. The reaction mixture was stirred at room temperature for 8 hrs (FIG. 5B, step 2). The PEG_{20 kDa}-RGD nanocarriers were purified on Sephadex G25 size-exclusion column using deionized (DI) water as eluent. The purified nanocarriers were obtained after freeze-drying. The nanocarriers were characterized using gel-permeation chromatography (GPC) and/or MALDI-TOF mass spectrometer.

### Synthesis and Characterization of 4-Arm and 8-Arm PEG-LA Nanocarriers.

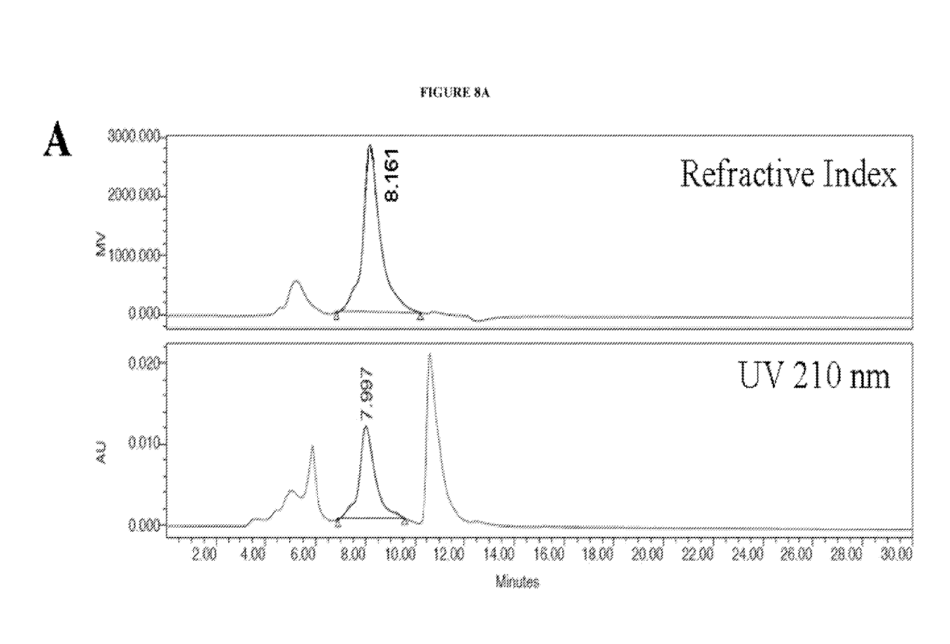

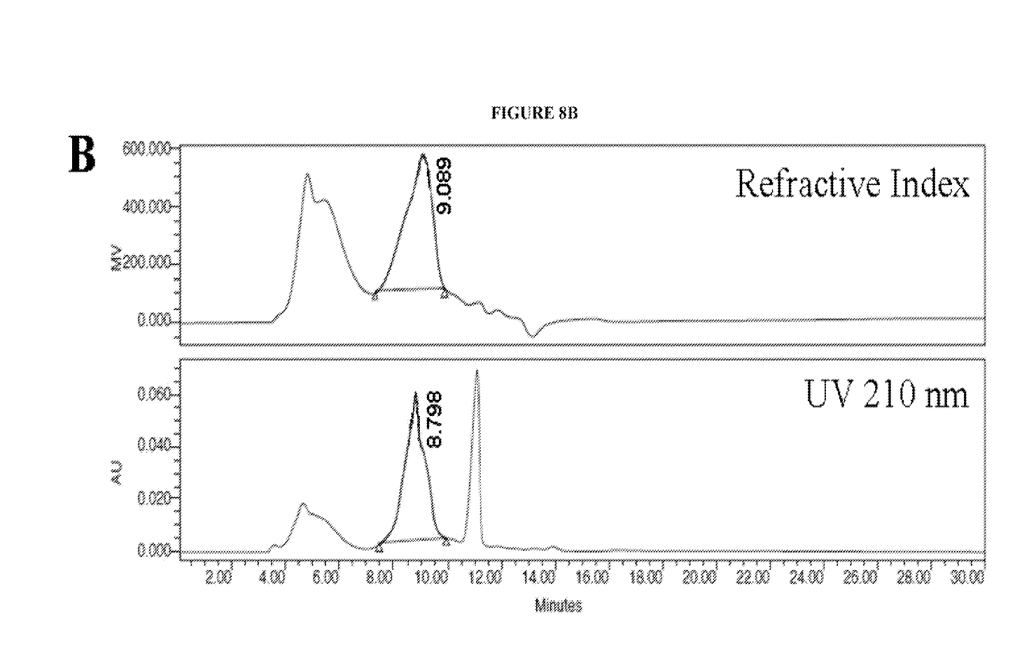

Multiple copies of lactic acid were attached to 4-arm and 8-arm PEG_{20 kDa}-SH polymers, via degradable thioester bond. (FIG. 7). Lactic acid was activated with N-hydroxysuccinimide in the presence of dicyclohexylcarbodiimide (DCC) to form N-hydroxysuccinimidyl ester of lactic acid. The ester was then reacted with four-arm PEG_{20 kDa}-SH and eight-arm PEG_{20 kDa}-SH at room temperature in the presence of 4-dimethylaminopyridine (DMAP). The pure nanocarriers were obtained by precipitation from ether followed by drying. Gel permeation chromatography (GPC) was used to determine the purity and molecular weight of PEG-LA nanocarriers. (FIG. 8). The retention time of four-arm and eight-arm PEG-LA nanocarriers were estimated from the GPC chromatograms as 8.6 and 9.09 min. The lactic acid loading efficiency of nanocarriers was estimated by quantifying their free thiol content by Ellman's assay. Ellman's assay showed 0.403 mM of lactic acid in 0.1 mM of 8-arm PEG-LA nanocarriers (four copies of lactic acid per molecule). Ellman's assay showed that the lactic acid loading efficiency was in the range of 21-85%.

### Synthesis and Characterization of PEG-LA Nanocarrier-Based Hydrogels.

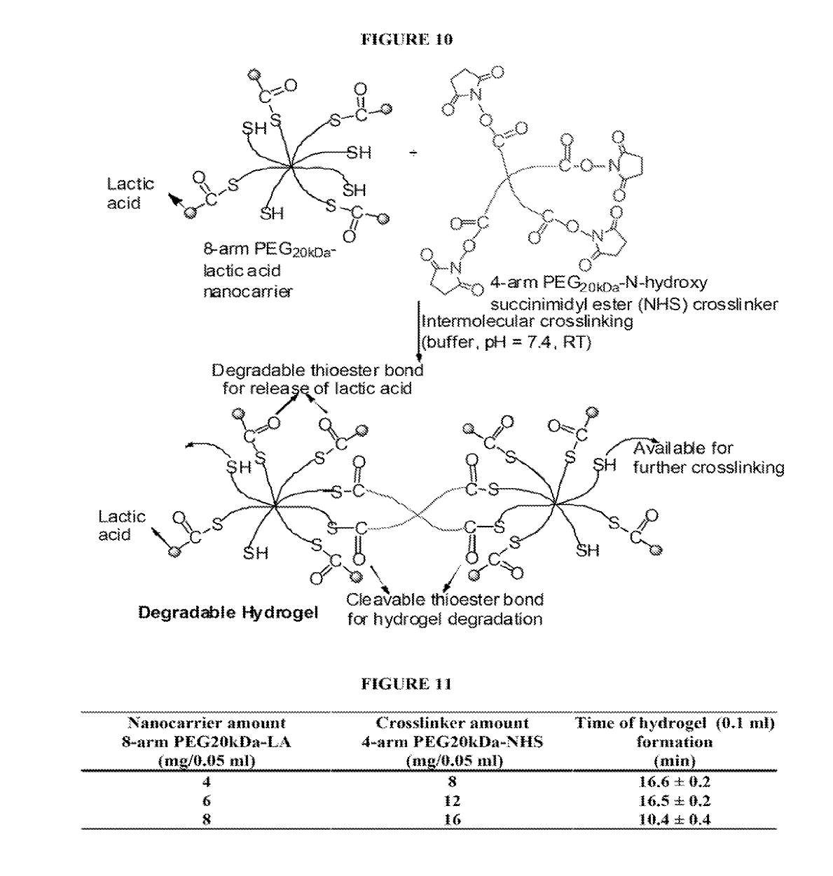

Hydrogels were prepared using degradable thioester crosslinks as follows: the 8-arm PEG-LA nanocarriers (4%, 6% and 8%; w/v) were mixed with varying amounts (4% to 16%) of 4-arm PEG_{20 kDa}-NHS crosslinker in sodium phosphate buffer (20 mM, pH 7.4) at room temperature, and the time of hydrogel formation was recorded. (FIGS. 10 and 11). The PEG-LA nanocarriers formed hydrogels with four-arm PEG-NHS crosslinker in ˜1.5 min

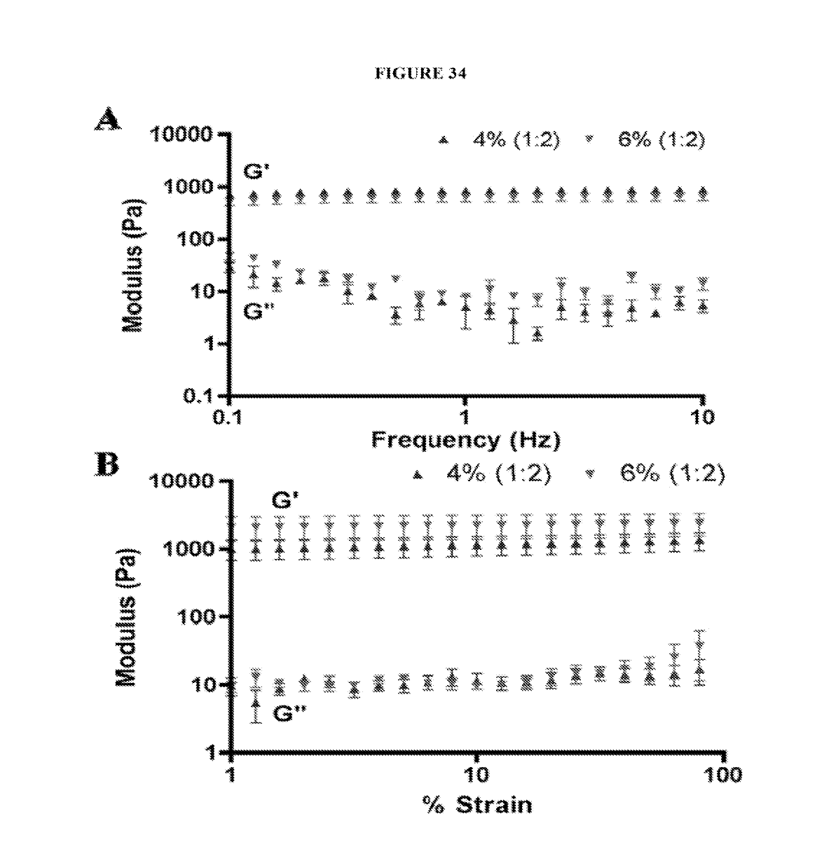

Rheological measurements were performed at 37° C. using a rheometer with parallel plate geometry (plate diameter: 20 mm, gap: 300 μm). PEG-LA hydrogels (4% and 6% w/v, 1:2) were allowed to form between the parallel plates at RT, before ramping the temperature up to 37° C. The elastic/storage modulus G′ and viscous/loss modulus G″ of the hydrogels were measured as a function of strain and frequency using dynamic oscillatory tests. First, a strain sweep test was performed at a constant frequency of 1 Hz, in order to determine the linear viscoelastic regime. Next, a frequency sweep test (0.1-1Hz) was carried out at a constant strain of 1%. All rheological measurements were done in triplicate and the mean±SEM reported. It was found that G′ was higher than G″ over the frequency range tested, indicating that the hydrogels were more elastic than viscous (FIG. 34). Moreover, the storage modulus only increased slightly with frequency and reached a plateau, indicating that the hydrogels can resist structural changes under strain.

Based on these results for PEG-LA hydrogels, the feasibility of crosslinking nanocarriers with other functionalities to form a hydrogel is demonstrated. It is expected that the hydrogels will possess the physical attributes of a good barrier membrane: they are highly dispersible providing extensive coverage of irregular surfaces; they possess sufficient viscosity to slow viral diffusion; and have outstanding elastic properties to withstand applied strain allowing the hydrogel barrier to remain intact during physical activity.

Preparation of Hydrogels with Passively Entrapped Lactic Acid.

Eight-arm PEG-SH (4%, 6% and 20%; w/v) with 200 μg of lactic acid and either 4-arm PEG_{20 kDa}-NHS (8% and 12%; w/v) or 8-arm PEG_{20 kDa}-NHS (20% w/v) were mixed in PB at room temperature. The time of formation of these hydrogels is provided in the table below.

Time of formation of hydrogels with passively entrapped
lactic acid; Mean ± S.D., n = 3

| 8-arm | 4-arm | Time of hydrogel |
|---|---|---|
| PEG_{20 kDa}-SH (mg) | PEG_{20 kDa}-NHS (mg) | formation (min) |
| 4 | 8 | 9.8 ± 0.2 |
| 6 | 12 | 6.6 ± 0.2 |
| 20 | 20 | 1.67 ± 0.1 |

Release of Lactic Acid from PEG-LA Nanocarriers, PEG-LA Nanocarrier-Based Hydrogels, and Hydrogels with Passively Entrapped Lactic Acid.

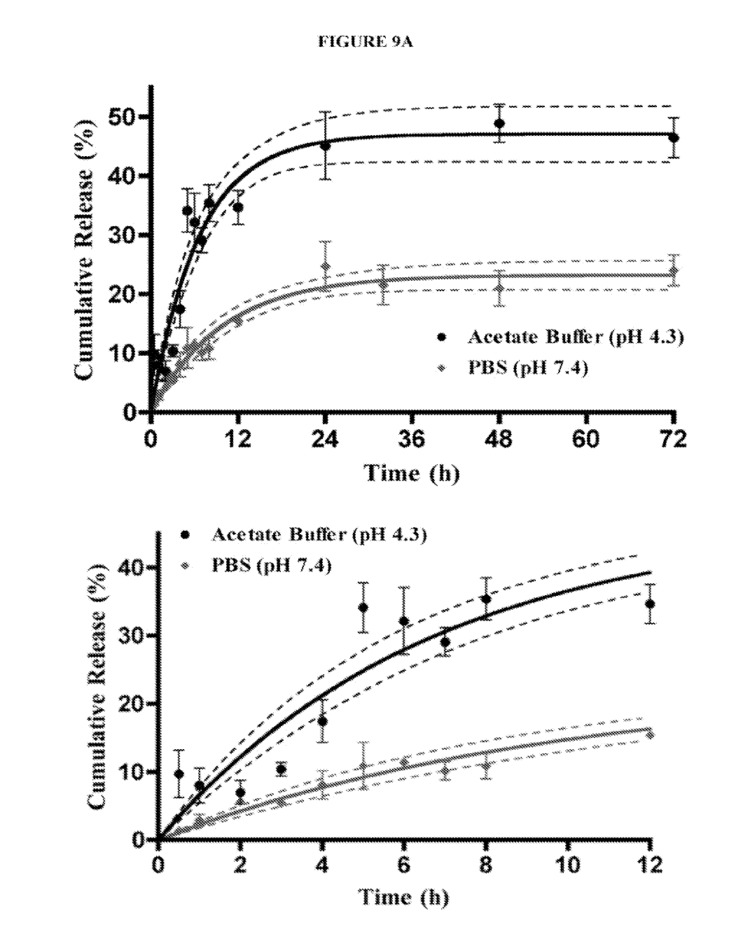

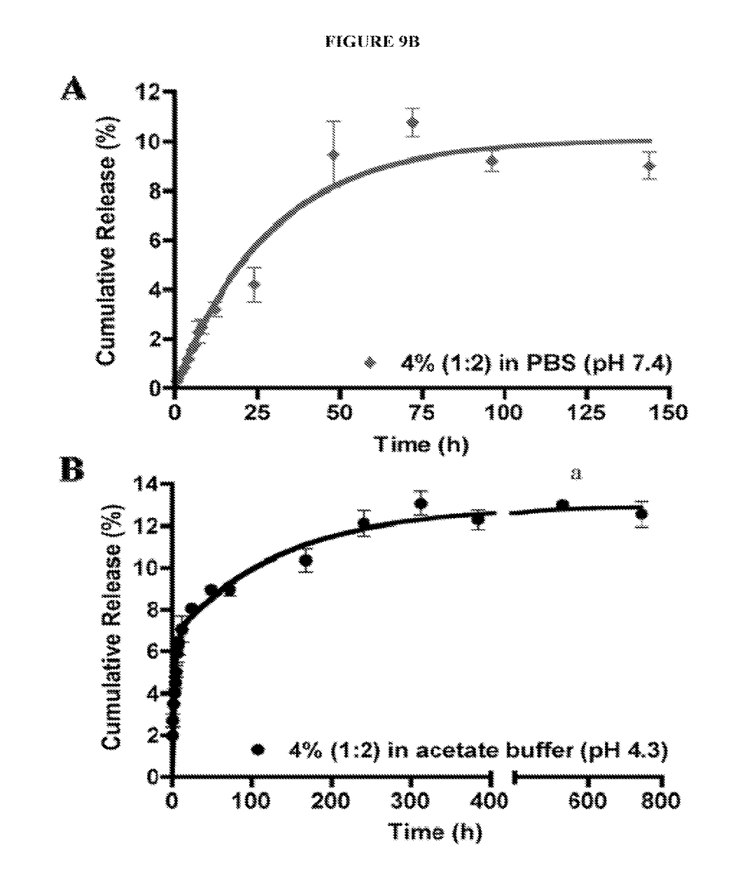

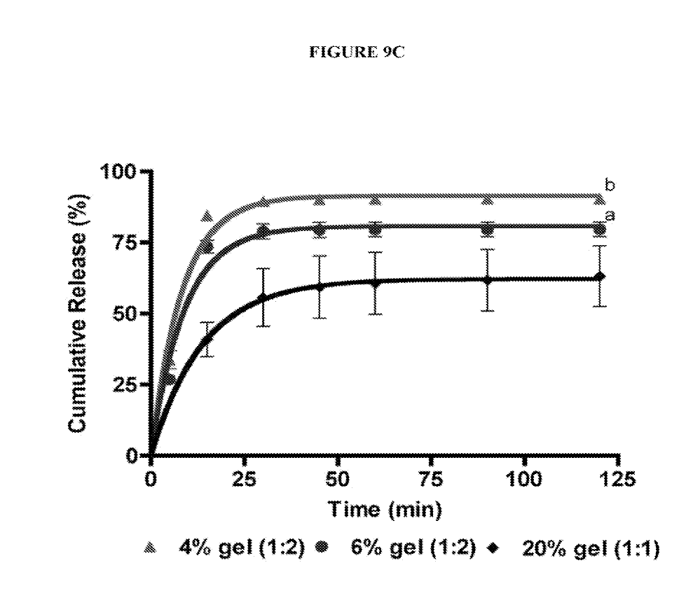

The 8-arm PEG-LA nanocarriers (1 mg/100 μl) were dissolved in sodium phosphate buffer (20 mM, pH 7.4). Dissolved 8-arm PEG-LA, PEG-LA hydrogel, and hydrogel with passively entrapped lactic acid was dialyzed against 3.6 ml PBS (10 mM, pH 7.4) or acetate buffer (pH 4.3) at 37° C. Aliquots (1 ml) were withdrawn at pre-determined time-intervals and the medium was replenished. A lactate assay kit (BioVision, Inc.) was used for quantifying the amount of lactic acid released, as per the manufacturer's protocol (O.D. at 570 nm). (FIG. 9A-9C). The release of lactic acid from nanocarriers was found to be pH-dependent; faster in acidic pH (acetate buffer, pH 4.3, half-life: 4.6 h) compared to physiological pH (PBS, pH 7.4, half-life: 6.8 h). Nanocarrier-based hydrogels offered a controlled release of lactic acid for several hours (t_{1/2}=20.03 h in PBS; t_{1/2}=93.11 h in acetate buffer).

### Hydrogel Swelling and Degradation Studies.

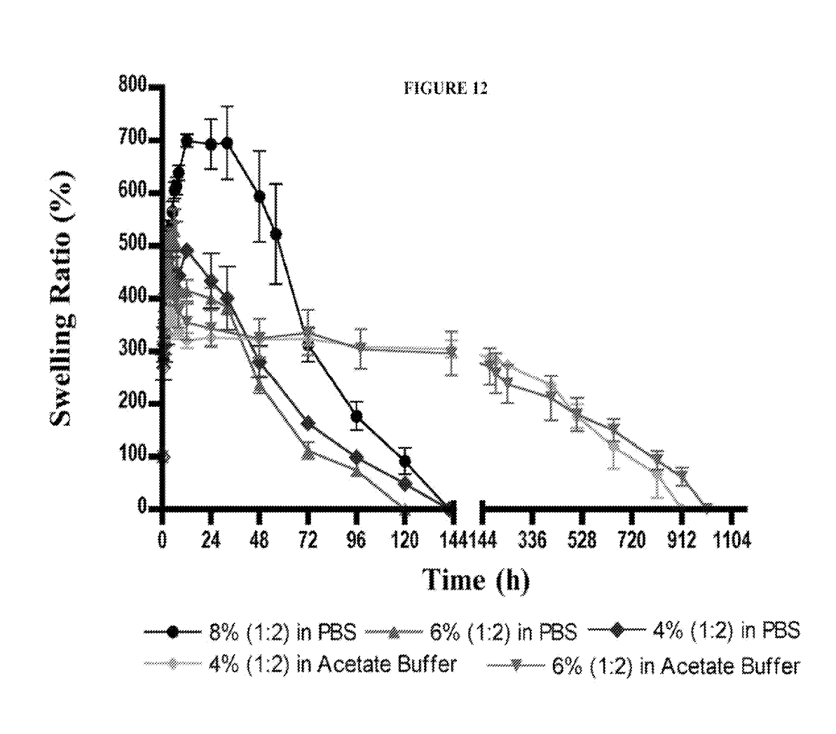

Hydrogels were weighed (W₀) and immersed in PBS (1.0 mL, 10 mM, pH 7.4) or acetate buffer (pH 4.3), and incubated at 37° C. The buffer was withdrawn at pre-determined time intervals and hydrogel weights were recorded (Wₜ). The swelling ratios were calculated as Wₜ/W₀×100, and plotted against time. (FIGS. 12a and 12b). Hydrogels were found to degrade <48 h in PBS and >5 days in VFS. PEG-LA nanocarrier-based hydrogels showed concentration-dependent swelling behavior (8%>6%>4%) in both PBS (pH 7.4) and acetate (pH 4.3) buffers.

### Hydrogel Swelling and Degradation Studies.

Hydrogels were weighed (W₀) and immersed in PBS (1.0 mL, 10 mM, pH 7.4) or acetate buffer (pH 4.3), and incubated at 37° C. The buffer was withdrawn at pre-determined time intervals and hydrogel weights were recorded (Wₜ). The swelling ratios were calculated as Wₜ/W₀×100, and plotted against time. (FIGS. 12A and 12B). Hydrogels were found to degrade <48 h in PBS and >5 days in VFS. PEG-LA nanocarrier-based hydrogels showed concentration-dependent swelling behavior (8%>6%>4%) in both PBS (pH 7.4) and acetate (pH 4.3) buffers.

### Polyanionic Stably Aggregated Nanoparticles to Bind HIV Virions.

The feasibility of producing stably aggregated PEG nanogel particles was determined. PEG nanogels (˜20 nm) were made using a one step synthetic procedure. As shown in FIG. 13, a 20 kDa 8-arm PEG-SH nanocarrier was crosslinked using a HVBS linker at various stoichiometries (1:1, 0.5:1, and 0.8:1). Using a variety of different conditions (sonication, surfactants, stirring rate and duration), these nanoparticles were stably aggregated in various sizes ranging from 1 to hundreds of microns. As can be seen in the TEMs in FIG. 14, nanogels (Panel A), stable nanogel aggregates in the low micron size range (Panel B) and aggregates in the high micron size range (Panel C) were produced. Particles that will be loaded into the hydrogel matrix were produced.

Subtilosin A: a Safe Microbicidal Protein from Bacillus amyloliquefaciens.

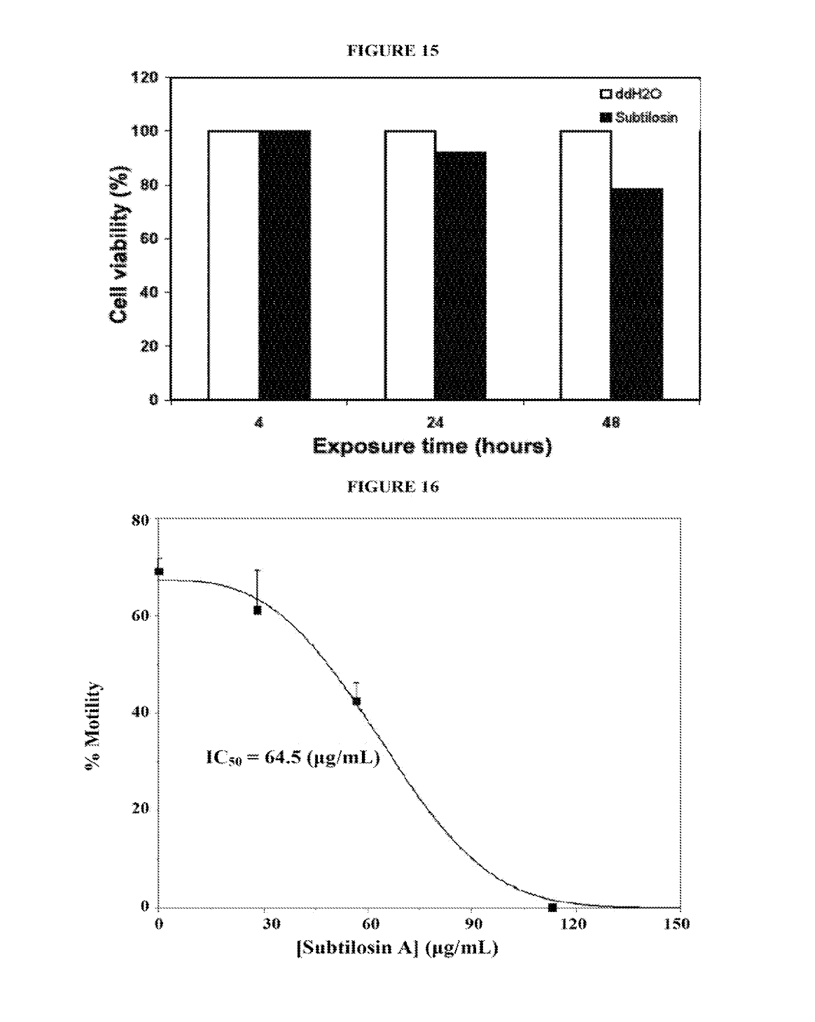

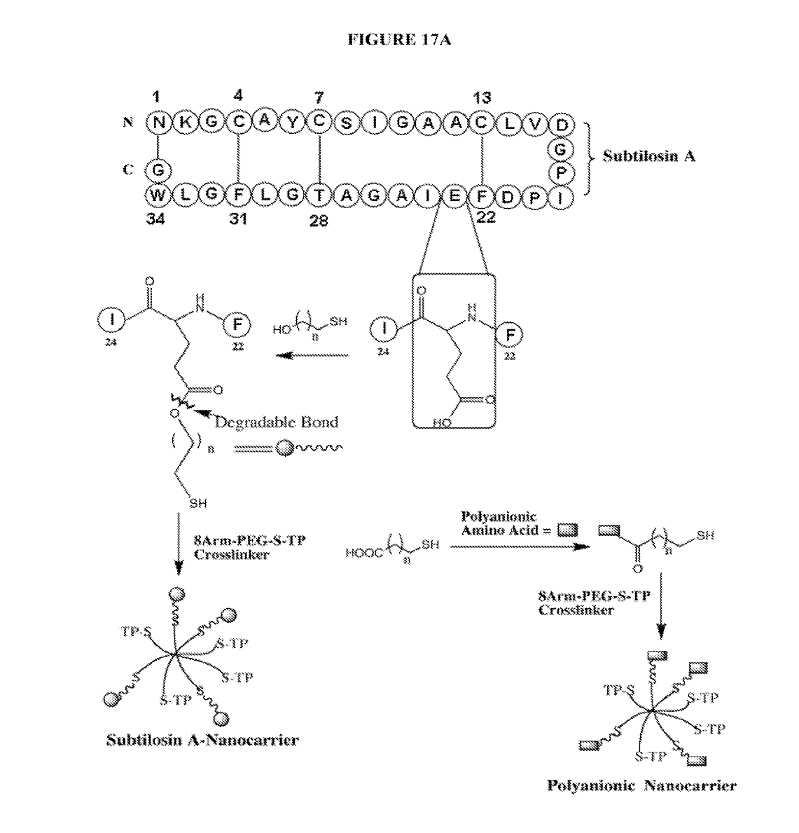

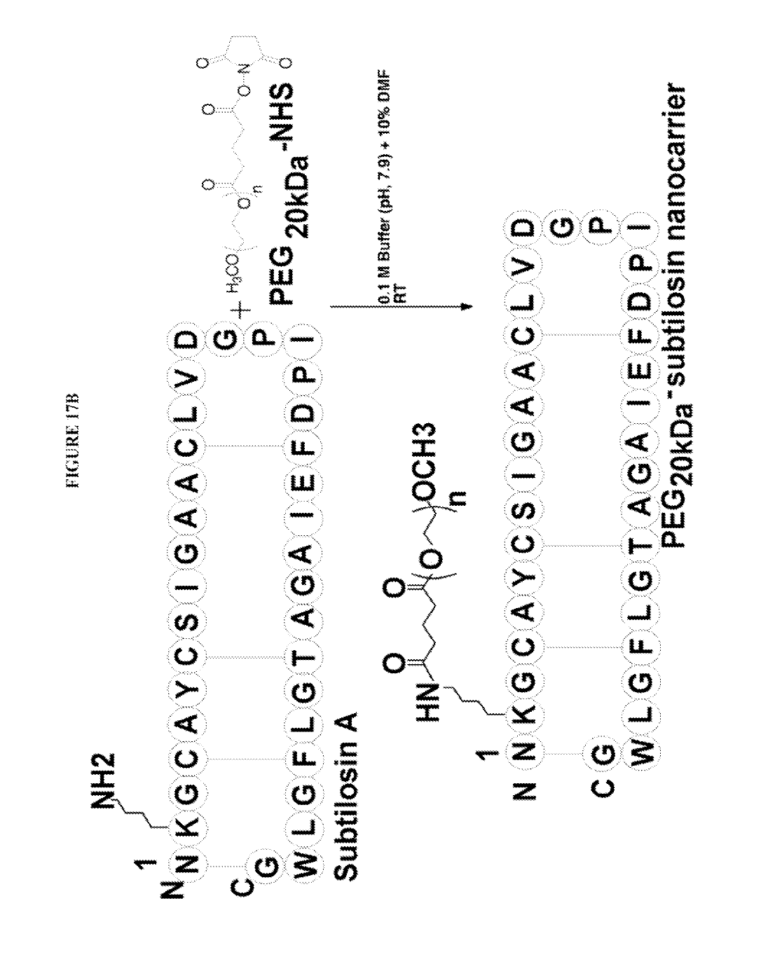

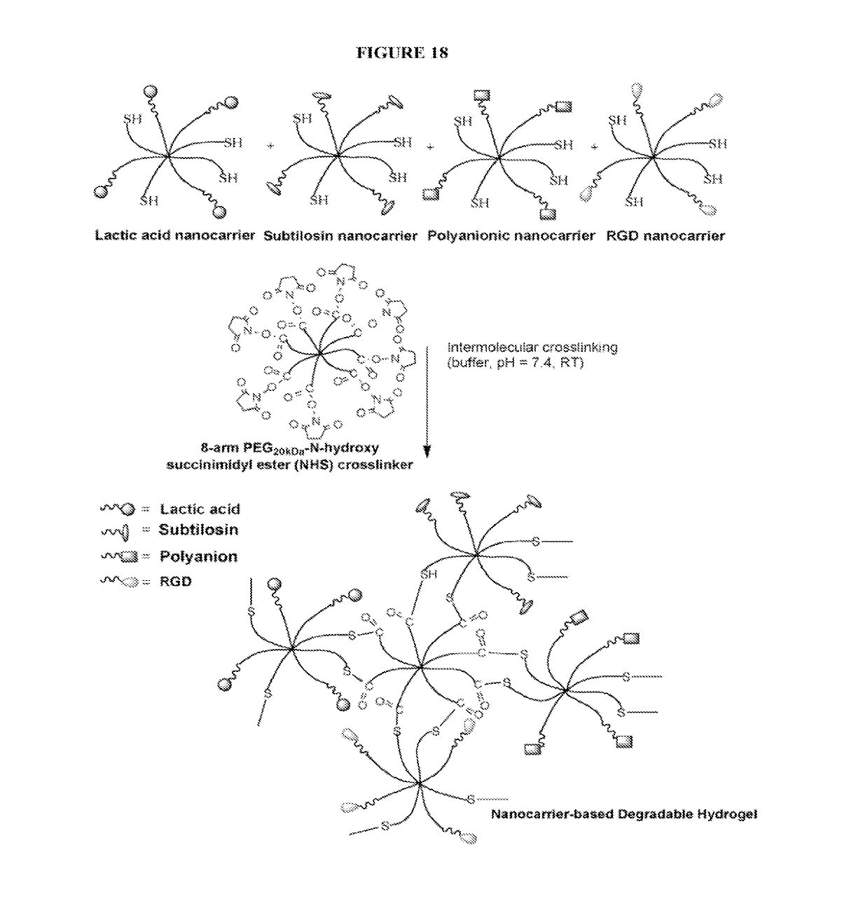

The antimicrobial activity of subtilosin against G. vaginalis makes it a prime candidate for inclusion in the microbicide hydrogel of the present invention. The toxicity of subtilosin was examined using the EpiVaginal Tissue Model™ (MatTek Corp.), which utilizes human vaginal ectocervical cells that are free from viral, yeast and bacterial infections. This three dimensional tissue model was exposed to subtilosin and other antimicrobial compounds to determine how prolonged exposure affects cell viability. Viability is measured as proportional to the breakdown of the yellow compound MTT to purple formazan by the ectocervical cells. After 24 hours, approximately 93% of the cells remained viable, with only a slight decrease to 73% viability after 48 hours (FIG. 15). This is in direct contrast to the results from the positive control, nonoxynol-9. This commonly used spermicidal agent, which has proven cytotoxicity, caused a 50% decrease in cell viability after only 4.9 hours.

### Subtilosin A: Spermicidal Microbicide.

Several microbicides under clinical development for the prevention of sexually transmitted infections (including those for which trials have recently been halted) have contraceptive properties (e.g., Pro2000, SAVVY, VivaGel, cellulose sulfate). However, none of those in development are spermicidal. Their contraceptive effects are mediated by effects on sperm function rather than cell death. While contraceptive activity in some cases is quite good (e.g., cellulose sulfate), it depends on the correct timing and placement of the product. A contraceptive microbicide that is truly spermicidal would not be as dependent on these variables, and would likely be more efficacious. We tested the effect of subtilosin on human sperm motility and the results are encouraging. This study was carried out by exposing whole semen to different concentrations of subtilosin. Thirty seconds after adding the compound to the semen, each sample was microscopically examined for sperm motility and forward progression. The subtilosin solution decreased the proportion of motile spermatozoa in a dose-dependent manner (FIG. 16). Motility ranged from 0% to 88% of controls. All samples with subtilosin had a reduction in motile spermatozoa as compared with the control samples (p<0.05, Newman-Keuls multiple range test). Forward progression is decreased by subtilosin in a dose-dependent manner The proportion of motile spermatozoa showing forward progression in the control samples exceeded 70%. This decreased to 50-70% in the presence of 50 μL of subtilosin, while 100 μL decreased forward progression to approximately 10%. At 200 μL, forward progression was absent and most of the sperm tails became coiled. Coiling of the tail is considered a sperm abnormality indicating damage to the plasma membrane.

Subtilosin Produced by B. amyloliquefaciens

### Bacterial Strains, Growth Conditions, and Culture Media

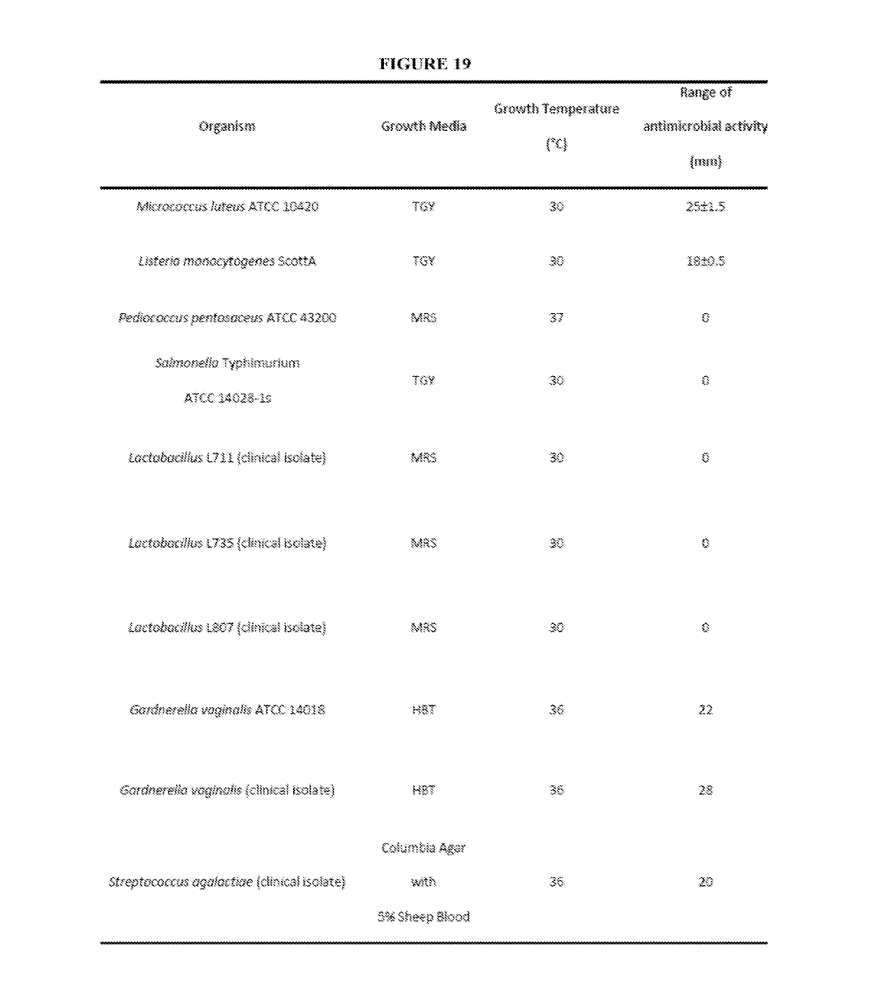

B. amyloliquefaciens was isolated from the yogurt-flavored cultured beverage Yogu Farm™ (JSL Foods, Los Angeles, Calif.) purchased from Hong Kong Market, New Brunswick, N.J., by aliquoting 1 ml of the product into 20 ml of MRS broth (Difco™, Detroit, Mich.). The culture was incubated for 48 hours at 37° C. in 5% CO₂ atmosphere without agitation. Inoculated plates were also incubated in the same conditions. Samples of the liquid culture were examined with phase microscopy to visualize basic cell characteristics. Culture samples were sent to the Laboratory for Molecular Genetics (Cornell University, Ithaca, N.Y.) for ribotyping and to Accugenix (Newark, Del.) for 16S ribosomal RNA (rRNA) analysis to confirm the identity of the unknown organism. Micrococcus luteus ATCC 10420, Listeria monocytogenes Scott A and Salmonella Typhimurium ATCC 14028-1s were grown in Tryptic Soy Broth supplemented with 0.6% Yeast Extract (Difco™) at 30° C. under aerobic conditions. Pediococcus pentosaceus ATCC 43200 was cultivated in MRS broth at 37° C. for 24 hours under aerobic conditions. Gardnerella vaginalis ATCC 14018 was grown on HBT agar (BD, Franklin Lakes, N.J.), while Streptococcus agalactiae (Group B Streptococcus) was grown on Columbia agar with 5% Sheep Blood (BD). Both organisms were incubated at 36° C. in 5% CO₂ atmosphere without agitation. The indicator strains used in well diffusion assays were obtained from either ATCC collections or as clinical isolates from the Rush Presbyterian Medical Center in Chicago, Ill. (FIG. 19).

### Sample Preparation

Cell-free supernatant (CFS) harvested from MRS broths was incubated for 48 hours at 37° C. in 5% CO₂ atmosphere (until approximately 106 CFU ml−1). Cells were removed from the culture by centrifugation (Hermle Z400K, LabNet, Woodbridge, N.J.) for 25 min at 4500×g and 4° C. Supernatants were filter-sterilized using 0.45 μm microfilters (Fisher, Pittsburgh, Pa.).

### Assay of Antimicrobial Activity

Well diffusion inhibition assays were conducted as described by Cintas, L. M., et al., “Isolation and characterization of pediocin L50, a new bacteriocin from Pediococcus acidilactici with a broad inhibitory spectrum,” Appl Environ Microbiol 61, 2643-48 (1995). with the following modifications. The efficacy of the B. amyloliquefaciens product at inhibiting the growth of various microorganisms was tested using CFS against MRS broth as a negative control and nisin (10 mg ml⁻¹) (Sigma, St. Louis, Mo., 2.5% bacteriocin preparation [106 IU g⁻¹] dissolved in ddH₂O) as a positive control. The indicator organism was grown overnight according to its specific growth requirements, with M. luteus used as a standard based on its known sensitivity to bacteriocins Pongtharangkul, T. and Demirci, A., “Evaluation of agar biodiffusion assay for nisin quantification,” Appl Microbiol Biotechnol 65, 268-72 (2004). Soft agar was made by adding 0.7% agar to either TGY or MRS; solid base plates were dried in a sterile hood for approximately 90 min prior to use in order to remove any extraneous moisture. To create an overlay, the indicator organism was added to the soft agar in a ratio of 100 μl bacterial culture per 10 ml soft agar (ca. 106 CFU ml⁻¹). From this mixture, 4 ml was overlaid onto each base plate and allowed to completely solidify. Pasteur pipettes were used to create 5 mm wells in the overlaid base plates. These wells were then allowed to dry for approximately 30 min. Then, 50 μl of each sample was added to the wells and allowed to freely diffuse for 45-60 min. All plates were then incubated overnight at the optimal growing conditions for the indicator organism (FIG. 19). The procedure for testing activity against the clinical isolates varied slightly from the previously described method. The indicator organism was inoculated as a lawn using a sterile swab, and after air-drying for 5 min, 17-mm wells were punched into the agar using a sterile glass test tube, and 400 μl of CFS was added. The plates were kept at room temperature for 2 h to allow for absorption of the supernatant, and then incubated overnight at 36° C. with 5% CO₂ atmosphere.

### Determination of Lactic Acid Concentrations

Lactic acid concentrations in the CFS were determined using a D-Lactic acid/L-Lactic acid test kit and according to the manufacturer's protocol (Roche Boehringer, Mannheim, Germany) After completing the steps of the protocol, the gathered data was applied to the provided equations in order to accurately calculate the quantities of each acid form in the sample.

### Enzymatic Digestion to Confirm Proteinaceous Nature of Antimicrobial Compound

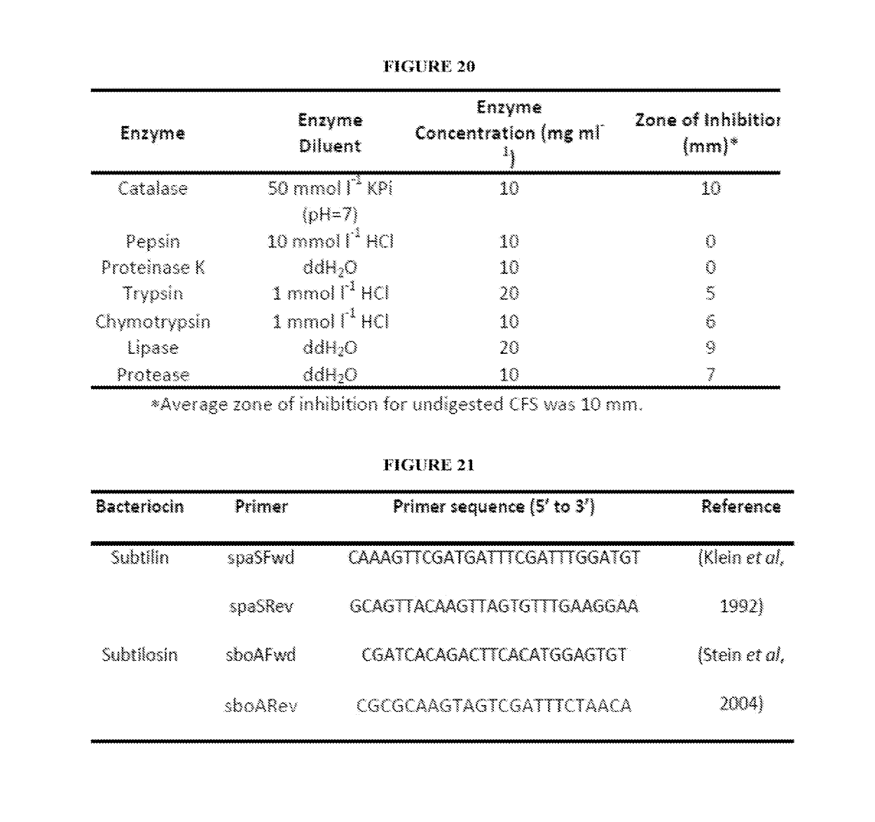

The CFS was exposed to seven different enzymes (Sigma; FIG. 20) overnight to determine the type of compound causing bacterial growth inhibition. Aliquots (250 μl) of the CFS were combined with equal volumes of the enzymes and the pH of the mixture and the incubation temperature were adjusted to those optimal for enzymatic activity. Two controls were used: (i) the enzyme mixed with sterile MRS media, and (ii) the CFS and enzyme diluent FIG. 20. After 24 h the pH of all samples was readjusted to ˜6 to attain maximum antimicrobial activity. Well diffusion assays were conducted in triplicate against the indicator Micrococcus luteus.

### Protein Visualization

SDS-PAGE was conducted using a Tris-Tricine gel made in a Bio-Rad casting apparatus (Bio-Rad, Hercules, Calif.). The gels were loaded with either 20 μl of marker or 200 μl of sample [1:1 sample+loading buffer (Bio-Rad)]. Nisin (10⁶ IU g⁻¹, 2 mg ml⁻¹) was used as the positive control. The procedure was conducted in 0.2 mol l⁻¹ Tris-base anode running buffer (pH=8.9) and 0.1 mol l⁻¹ Tris/0.1 mol l⁻¹ Tricine/0.1% SDS cathode running buffer (pH=8.25) in a Mini-Protean 3 (Bio-Rad) chamber with Power-Pac 300 power source (Bio-Rad).

Upon completion of electrophoresis, the gel was cut into identical halves; one half was treated for the overlay process while the other was used in the staining procedure. The overlay gel was fixed for 2 h in 100 ml of 10% acetic acid/20% isopropanol buffer, rinsed 3 times over 2 h in 100 ml ddH₂O, and stored overnight in ddH₂O at 4° C. (all steps occurred under rotation). The following day, it was laid onto a dried enriched TSA plate and overlaid with M. luteus. The staining gel was processed according to the manufacturer's Silver Stain protocol (Bio-Rad).

### Protein Purification

Using a stock overnight culture, B. amyloliquefaciens was inoculated (ca. 106 CFU ml⁻¹) and grown in 500 ml MRS under normal conditions. Cells were removed by centrifugation for 25 min. at 12120×g. The CFS was filter-sterilized as previously described. A nomogram was used to calculate the amount of solid ammonium sulfate needed to achieve 30% saturation, which was added to the solution incubated at 4° C. overnight while stirring. The following day, the precipitate was gathered by centrifugation as described above and re-dissolved in 20 ml ddH₂O. Activity of both the precipitate and the supernatant were tested in a well diffusion assay against M. luteus. The precipitate was used to conduct all further experiments and is designated as the “sample”.

Further purification of the 30% ammonium sulfate precipitate was achieved with Sep-Pak® Light C18 Cartridges (Waters, Milford, Mass.) to separate the protein of interest based on an assumed hydrophobic nature. In each instance, 0.5 ml of liquid was passed through the column at a flow rate of 0.2 ml min⁻¹. The cartridge was initially rinsed with 0.5 ml 100% methanol and equilibrated by four 0.5 ml washes with ddH₂O to remove any traces of the methanol. Following the water washes, the sample was loaded onto the cartridge and the flow-through was collected. This was followed by another four 0.5 ml washes with ddH₂O, with each fraction collected individually Immediately after the water washes, the column was washed sequentially with 1 ml of 50%, 70%, 90% and 100% methanol, and individual 0.5 ml fractions were collected. Antimicrobial activity was confirmed by the well diffusion assay.

### Effect of Temperature and pH on Antimicrobial Activity

The ability of the compound to retain activity under elevated temperatures was tested by incubating the sample at a given temperature for 0-60 min. After each time point 200 μl was aliquoted and used to create 2-fold serial dilutions in ddH₂O. Each dilution was used in a well diffusion assay; the reciprocal value of the lowest dilution that maintained activity is considered the protein concentration in arbitrary units (AU) ml⁻¹.

The level of antimicrobial activity of the sample was tested at varying pH levels. The pH of the solution was adjusted to fall within the range of 2-10 using either 3 mol l⁻¹ HCl or NaOH. The samples were incubated at room temperature for 1 min. before conducting a well diffusion assay against M. luteus.

### Genetic Analysis

DNA was extracted from overnight cultures of B. amyloliquefaciens and B. subtilis ATCC 6633 using the Promega Wizard SV Genomic DNA Kit (Promega Corp, Madison, Wis.) with the following modifications. Cells were harvested from the culture (2×1.5 ml) in a microfuge tube at 13,000×g for 3 min. and resuspended in 382 μl 0.5 mol l⁻¹ EDTA (pH 8.0). To this, 100 μl of lysozyme (20 mg ml⁻¹), 10 μl proteinase K (20 mg ml⁻¹) and 8 μl mutanolysin (2.5 U μl⁻¹) was added. The mixture was incubated for 60 min. at 37° C., following which 200 μl of nuclei lysis solution and 5 μl RNase A were added, and incubated for 20 min. at 65° C. Two hundred-fifty μl of lysis buffer was immediately added, and DNA was subsequently purified using the provided spin columns according to the manufacturer's specifications and eluted in 100 μl nuclease-free water.

Polymerase chain reactions (PCRs) were performed to assess the relatedness between the bacteriocin produced by B. amyloliquefaciens and the B. subtilis products subtilin and subtilosin. Primers (listed in FIG. 21) were designed using the B. subtilis genome (GenBank Accession #AJ430547) to specifically recognize the functional genes of subtilin (spaS) and subtilosin (sboA). Genomic DNA from B. amyloliquefaciens and B. subtilis ATCC 6633 was added to a master mix consisting of each primer, nucleotides, buffer and HotMaster Taq (Eppendorf, Hamburg, Germany) PCR was conducted using an Applied Biosystems GeneAmp PCR System 2400 apparatus (Applied Biosystems, Foster City, Calif.) under the following parameters: denaturation for 30 s at 94° C., annealing for 30 s at 55° C. (spaS) or 50° C. (sboA), and elongation for 1 min. at 65° C. for a total of 30 cycles. PCR products were sequenced using ABI Prism 3730xl DNA analyzers (GeneWiz, Inc., South Plainfield, N.J.), and the resulting sequences were analyzed using the Vector NTI software suite of programs (Invitrogen, Carlsbad, Calif.). The sequence obtained for B. amyloliquefaciens has been submitted to GenBank under the accession no. EU105395.

### Characterization of Unknown Isolate

While the Yogu Farm™ beverage was purported to contain Lactobacillus cultures, B. amyloliquefaciens was the only organism recovered from four individual lots of the product. Phase microscopy of each sample of bacterial growth revealed a single organism that was a very motile endospore-producing bacillus. On solid agar, the colonies tended to spread quickly into lawn formation, with an extremely wrinkled texture. The organism appeared to secrete a thick, opaque slime from the colonies, which was later revealed to be a byproduct of the starch-hydrolyzing enzyme amylase. Ribotyping and 16S rRNA analyses determined the bacterium to be Bacillus amyloliquefaciens, a closely related species to Bacillus subtilis.

### Range of Antimicrobial Activity

The CFS of a B. amyloliquefaciens culture was determined to have antimicrobial activity against a wide range of bacterial species, including the pathogens L. monocytogenes, G. vaginalis and S. agalactiae. There was no activity against several strains of vaginal probiotic Lactobacilli also gathered from the clinical setting (FIG. 19).

### Determination of Lactic Acid Concentrations

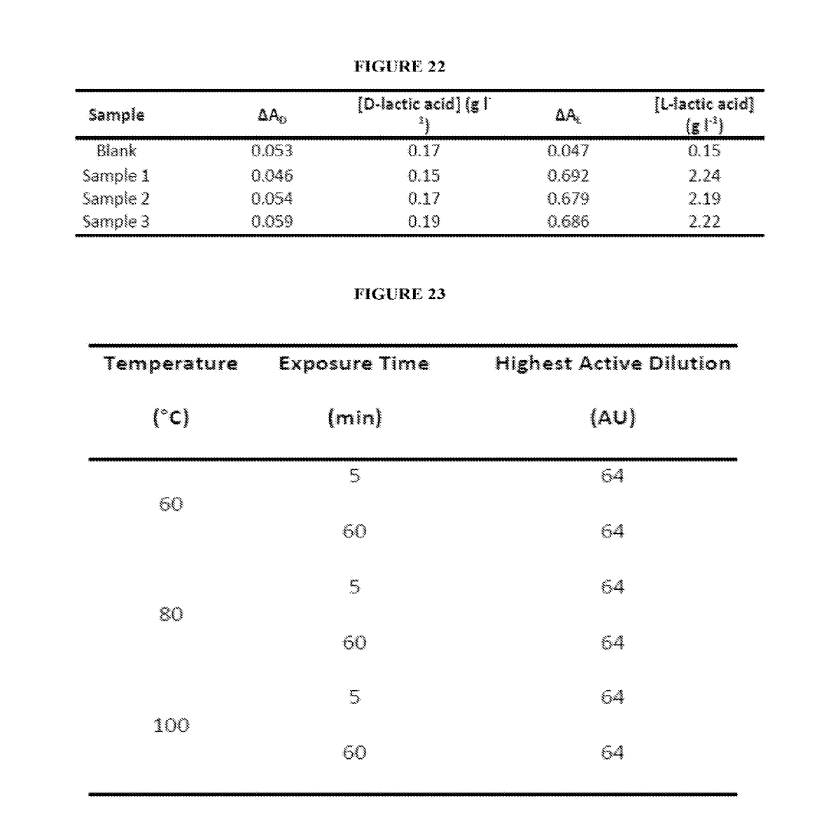

Using equations provided by the manufacturer's protocol, it was determined that B. amyloliquefaciens produced very low levels of both D- and L-lactic acid in three separately conducted assays. Calculations revealed that there was an average of 0.17 g l⁻¹ D-lactic acid per sample, a value equal to that of the tested blank. The average concentration of L-lactic acid rose to 2.22 g l⁻¹, which was slightly higher than the blank's concentration of 0.15 g l⁻¹ (FIG. 22). The very low basal concentrations of both forms of lactic acid suggest they do not play a significant role in microbial inhibition, and that all detected activity may be attributed to the bacteriocin.

### Effect of Enzyme Digestion, Temperature and pH on Antimicrobial Activity

Inhibition assays revealed that activity was completely lost in the presence of pepsin and proteinase K, and significantly decreased by trypsin and chymotrypsin, confirming the proteinaceous nature of the compound (FIG. 20). Exposure to increasingly high temperatures had no apparent effect on the protein, with activity still present (64 AU) after the sample had been heated for 60 min. at 100° C. (FIG. 23). There was also no reduction in activity at any of the pH values ranging from 2-10, despite the fact that the pH of the CFS was typically neutral (˜6.5) (data not shown).

### Protein Purification

The protein was fully precipitated out of solution at 30% ammonium sulfate concentration, and the presence of the bacteriocin was confirmed on SDS-PAGE gels with a large zone of inhibition in the overlay portion corresponding to the known size of subtilosin (data not shown).

Inhibition assays indicated that the protein was solely and completely eluted from the columns by 90% methanol. They also confirmed activity was wholly due to the antimicrobial peptide and not background activity from the methanol.

### Genetic Analysis

PCR analysis showed B. amyloliquefaciens to be negative for the functional gene encoding subtilin (spaS), but positive for the functional gene encoding subtilosin (sboA). The DNA sequence of the PCR product amplified from B. amyloliquefaciens was compared to that from B. subtilis ATCC6633, and was shown to be 91.7% identical. There were only three base pair changes in sboA, none of which affected the amino acid sequence of the protein. A homolog of sboX (95% identical), a gene which putatively encodes a bacteriocin-like substance and overlaps sboA, was also identified. The gene encoding YwiA (albA) is downstream of the gene encoding SboA, and is believed to have a role in the posttranslational modifications of subtilosin. Due to the overwhelming similarity of the two gene products, the sequence preceding the gene and the intergenic sequence were compared, and found to be 95.6% and 85% similar, respectively.

### Analysis of Anti-Microbial Activity

### Bacterial Strains and Growth Conditions.

Stock cultures of G. vaginalis ATCC 14018 were kept at −80° C. in BHI broth (Difco, Sparks, Md.) supplemented with 3% horse serum (JRH Biosciences, Lenexa, Kans.) and 15% glycerol. Cultures of G. vaginalis were grown anaerobically in BHI broth + 3% horse serum at 37° C. without shaking. B. amyloliquefaciens cultures were grown overnight in MRS broth (Difco) at 37° C. without shaking. The initial cultures were subcultured multiple times before use in experimental testing.

### Preparation of Antimicrobial Solutions.

The partially purified preparation of subtilosin was prepared as described in Sutyak et al., “Isolation of the Bacillus subtilis antimicrobial peptide from the dairy product-derived Bacillus amyloliquefaciens,” J. Appl. Microbiol. 104: 1067-74 (2007). Nisin (Sigma-Aldrich, St. Louis, Mo.; 100 AU/mL) was prepared according to the protocol given by Turovskiy et al., “Lactocin 160, a bacteriocin produced by vaginal Lactobacillus rhamnosus, targets cytoplasmic membranes of the vaginal pathogen, Gardnerella vaginalis,” Probiotics Antimicrob. Proteins 1:67-74 (2009).

### ATP Efflux Assay.

The effect of subtilosin on ATP depletion in G. vaginalis cells was assessed by the previously established bioluminescence method (Guihard et al., “Phosphate efflux through the channels formed by colicins and phage T5 in Escherichia coli cells is responsible for the fall in cytoplasmic ATP,” J. Biol. Chem. 268: 17775-80 (1993)) and modifications of Turovskiy et al. using an ATP Bioluminescent Assay Kit (Sigma-Aldrich) and a Luminoskan™ single-tube luminometer (Labsystems, Helsinki, Finland). This kit correlates ATP release with relative fluorescence as a result of oxidation of the D-luciferine molecule by the firefly luciferase enzyme in the presence of ATP and Mg2⁺. G. vaginalis cells were grown overnight in 15 mL BHI broth supplemented with 3% horse serum to an OD₆₆₀≈0.6. Once they reached the appropriate growth stage, cells were centrifuged for 15 min. at 4500 g (Hermle Z400K; LabNet, Woodbridge, N.J.) at room temperature, and then washed once with 50 mmol/L MES buffer (pH 6.5). The cells were then maintained at room temperature for 5 min. prior to an energization period, in which the cells were resuspended in half their original volume of 50 mmol/L MES buffer (pH 6.5) with 0.2% glucose and held at room temperature for 20 min. Following energization, the cells were collected by centrifugation under the aforementioned conditions and resuspended in half their original volume in 50 mmol/L MES buffer (pH 6.5). This suspension was aliquoted in 100 μL volumes into sterile 1.5 mL microcentrifuge tubes, to which 20 μL of the appropriate treatment was added. Subtilosin was used at a final concentration of 2 μg/mL, while the positive control (bacteriocin nisin) reached a final concentration of 1.5 μg/mL, as per Winkowski et al., “Correlation of bioenergetic parameters with cell death in Listeria monocytogenes cells exposed to nisin,” Appl. Envrion. Microbiol. 60:4186-88 (1994). Subtilosin diluent (ddH₂O) and nisin diluent (0.02M hydrochloric acid, pH 1.7) were used as negative controls. Each sample then remained at room temperature for 5 min. prior to recording bioluminescent measurements.

The total ATP concentration in G. vaginalis cells was measured by combining 20 μL of the final cell suspension with 4.9 mL ice-cold ddH₂O and 80 μL DMSO. DMSO was chosen for its known ability to completely lyse bacterial cells, thus releasing all intracellular ATP. The data obtained for the negative controls were extremely uniform, allowing all other results to be normalized to their average and expressed as a percentage value.

Effect of Subtilosin on Proton Motive Force (PMF) in G. vaginalis.

### ΔΨ Dissipation Assay.

The ability of subtilosin to affect the transmembrane electric potential (ΔΨ) of G. vaginalis cells was assessed according to the protocol given by Sims et al., “Studies on the mechanism by which cyanine dyes measure membrane potential in red blood cells and phosphatidylcholine vesicles,” Biochemistry 13: 3315-30 (1974) and the modifications outlined by Turovskiy et al.

Briefly, G. vaginalis cells were grown as previously described to an OD₆₀₀ of 0.6, harvested, then washed once and resuspended in 1/100 of their original volume of fresh medium. The ΔΨ of the cells was monitored as a function of the fluorescent intensity of the probe 3,3′-dipropylthiadicarbocyanine iodide [lDiSC₃] (Molecular Probes, Eugene, Oreg.) at 22° C. using a PerkinElmer LS-50B spectrofluorometer (PerkinElmer Life and Analytical Science, Inc., Boston, Mass.) with a slit width of 10 nm and excitation and emission wavelengths of 643 and 666 nm, respectively. Initially, 5 μL of probe was added to 2 mL of fresh BHI broth supplemented with 3% horse serum in quartz cuvettes (10 mm light path) at a final concentration of 5 μmol/L. This was followed by addition of 20 μL of cell suspension, which caused an immediate decrease in fluorescence. Once the signal had equilibrated, the cells were exposed to 2 μL of 5 mM nigericin (Sigma) in order to convert the ΔpH into ΔΨ. After stabilization of the signal, subtilosin, the positive control nisin, or the negative control nisin diluent was added. Finally, any remaining ΔΨ was dissipated by the addition of 2 μL of 2 mmol/L valinomycin (Sigma).

### ΔpH Dissipation Assay.

The ability of subtilosin to affect the transmembrane pH gradient (ΔpH) of G. vaginalis cells was analyzed according to the protocol given by Molenaar et al., “Continuous measurement of the cytoplasmic pH in Lactococcus lactis with a fluorescent pH indicator,” Biochim Biophys. Acta 1115: 75-83 (1991) and the modifications described by Turovskiy et al.

Initially, G. vaginalis cells were grown overnight to an OD₆₀₀ of 0.6, harvested, then washed twice and resuspended in a hundredth of their original volume of 50 mmol/L potassium phosphate buffer (PPB, pH 6.0). The cells were then exposed for 5 min. to the pH sensitive probe BCECF-AM (MP Biomedicals, Inc., Solon, Ohio) at ambient temperature to allow the probe to diffuse into the cytoplasm. Following exposure, the cells were washed twice with 1 mL of 50 mmol/L PBS (pH 6.0) and resuspended in 200 μL of the same. To measure dissipation of the transmembrane pH gradient, quartz cuvettes containing 2 mL of PPB (pH 7.0) were treated with 10 μL of the cell suspension. Fluorescence was read using a PerkinElmer LS-50B spectrofluorometer with slit widths of 5 nm for excitation and 15 nm for emission, and wavelengths of 502 and 525 nm, respectively. After signal stabilization, the cells were energized with 4 μL of 2.2 mmol/L glucose; the fluorescence subsequently rises as a result of an increase in intracellular pH. After again allowing for the signal to even out, 2 μL of 5 μmol/L valinomycin was added to convert the ΔΨ component of the PMF into ΔpH. The cells were then treated with either subtilosin, the positive control (nisin), or the negative control (nisin diluent). Two μL of 2 μmol/L nigericin was added to dissipate any remaining ΔpH.

Subtilosin Causes an Efflux of ATP from G. vaginalis Cells.

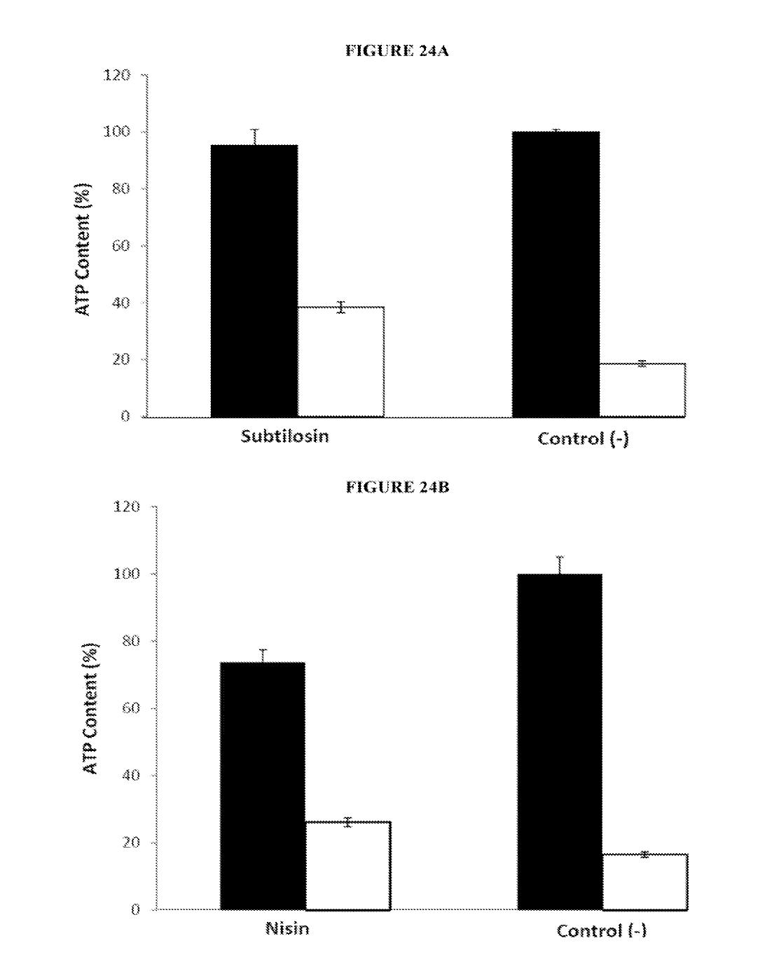

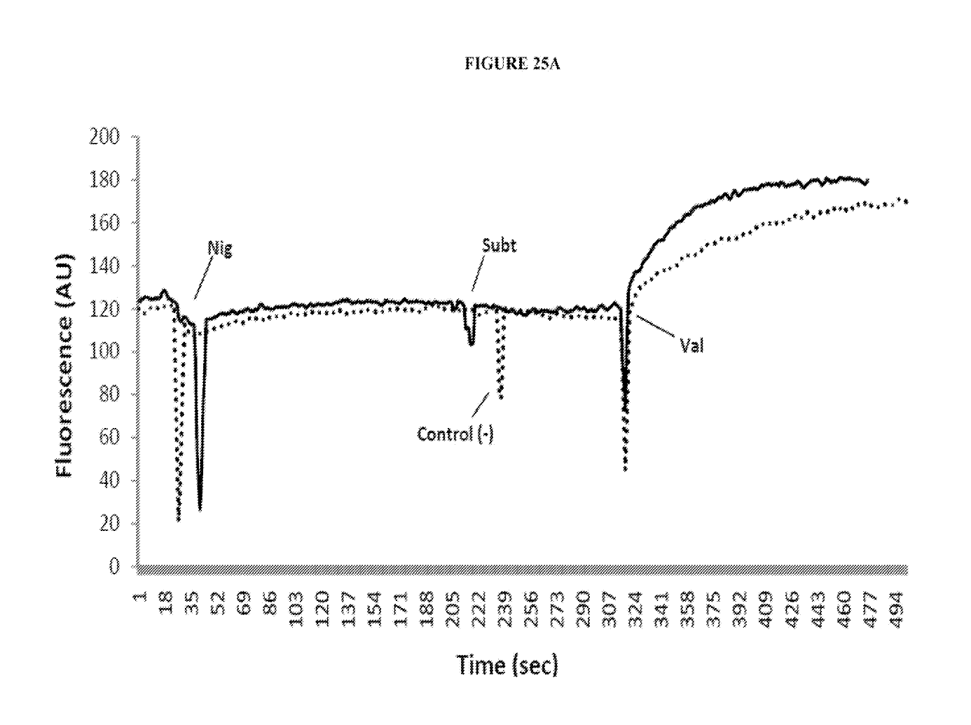

The effect of subtilosin on intracellular ATP levels in G. vaginalis cells was assessed as a function of bioluminescence, via the oxidation of the luminescent D-luciferine molecule by luciferase in the presence of extracellular ATP and Mg²⁺. In FIGS. 24A and 24B, closed bars represent the total ATP content (intracellular + extracellular), while open bars represent extracellular ATP. Subtilosin (24A) caused an efflux of ATP approximately 1.5-fold higher than that of nisin (24B) and 2-fold higher than the negative control. By contrast, the positive control (nisin) did not cause an efflux of ATP but instead triggered internal hydrolysis of the molecule, evidenced by a decrease in the luminescence in the total ATP sample (FIG. 24B). Total ATP levels for nisin (24B) were 20% lower than that of subtilosin (24A) and both negative controls (24A, 24B), indicating intracellular hydrolysis of ATP. It was not possible to determine the effect of exposure to subtilosin and the controls past the single 5 min. time point as the fastidiously anaerobic G. vaginalis cells poorly tolerated prolonged aerobic conditions (data not shown).

Subtilosin has No Effect on G. vaginalis Transmembrane Dlectrical Potential (ΔΨ).

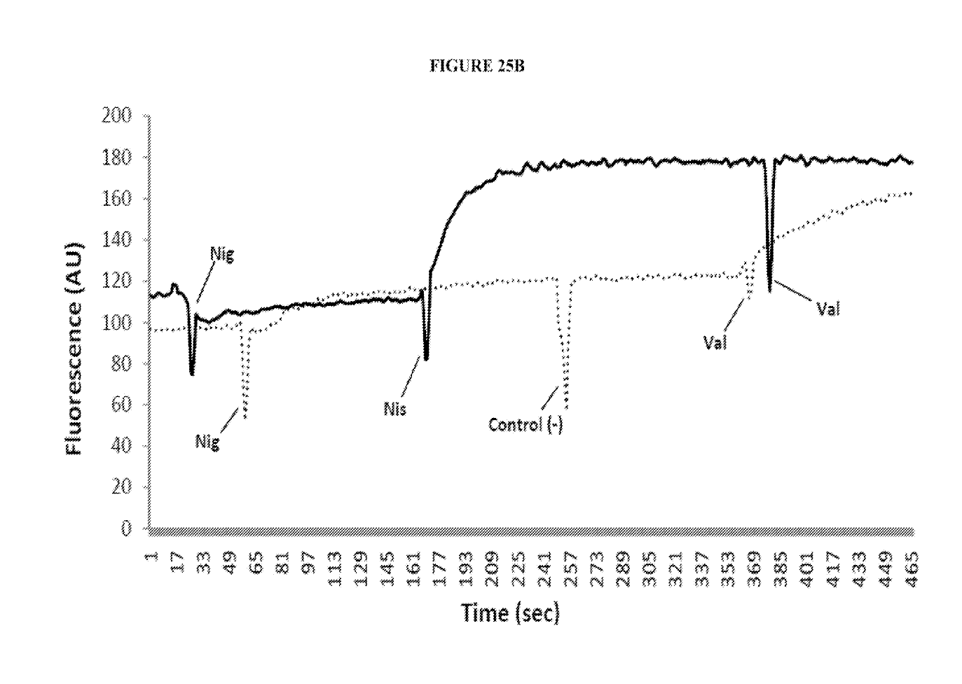

The ability of subtilosin to dissipate the transmembrane electrical potential (ΔΨ) in G. vaginalis cells was observed using the fluorescent probe 3,3′-dipropylthiadicarbocyanine iodide [DiSC₃]. The ionophore nigericin (a K⁺/H⁺ exchanger) was added to the G. vaginalis cells in growth medium in order to convert the ΔpH to ΔΨ. The addition of nisin caused an instantaneous increase in the fluorescent signal of the probe as a result of the cellular membrane being depolarized by the bacteriocin (FIG. 25B). Subsequent introduction of the ionophore valinomycin had little effect, indicating nisin caused a complete collapse of this PMF component. However, the addition of subtilosin or the negative controls (nisin diluent and ddH₂O) did not cause an elevation in the probe's fluorescence, signifying they had no effect on the ΔΨ (FIGS. 25A,B). For both nisin diluent and subtilosin, subsequent addition of valinomycin fully depleted the ΔΨ, resulting in a fluorescence increase comparable to that seen after the addition of nisin (FIGS. 25A,B). Unlike the positive control nisin, which caused a complete dissipation of the ΔΨ, subtilosin does not cause G. vaginalis cell damage by depleting this component of the PMF.

### Subtilosin Causes an Immediate Depletion of the Transmembrane pH Gradient (ΔpH).

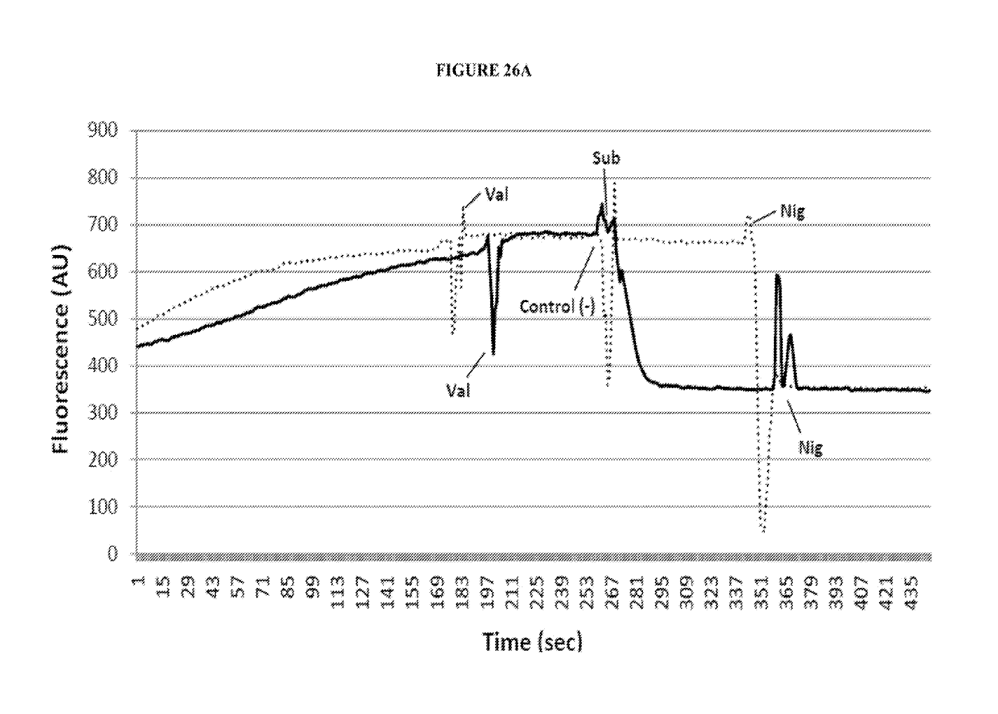

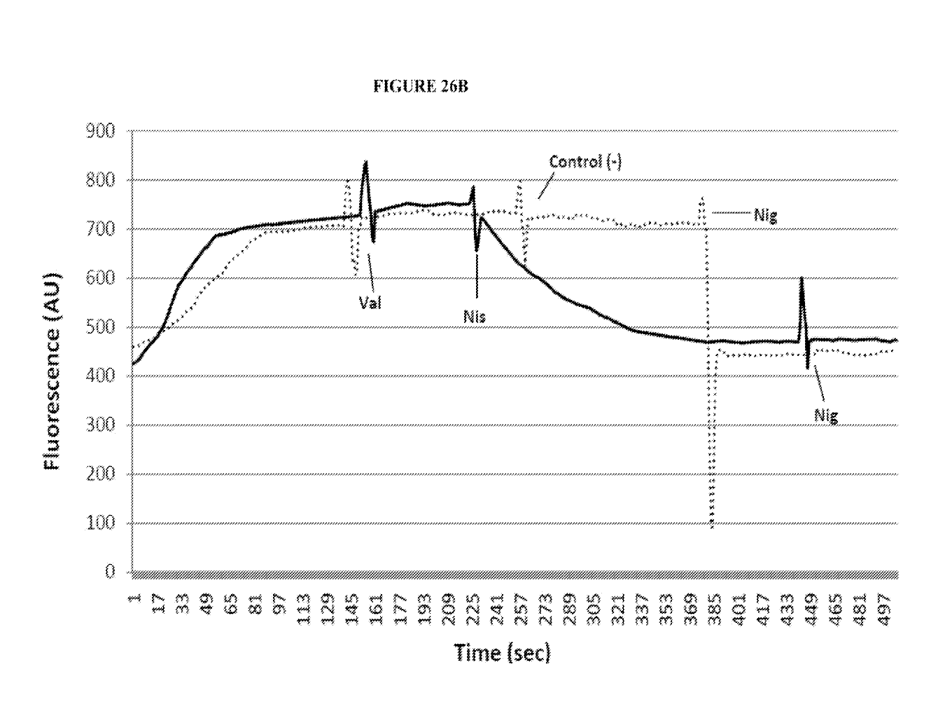

Cells were energized with 2.2 mM glucose at start of fluorescence readings. Two μmol/L valinomycin (Val) was used to transform the ΔΨ of the PMF into ΔpH Addition of subtilosin caused an instant drop in the signal intensity of the pH dependent, fluorescent probe BCECF-AM, indicating an immediate intracellular decrease in pH in the G. vaginalis cells (FIG. 26A). Nisin also caused a decrease in the fluorescent signal, although at a slower, more gradual rate (FIG. 26B). Since the assay buffer was designed to have a pH lower than the intracellular pH of G. vaginalis cells (39), the decrease in intracellular pH is due to a depletion of the ΔpH. Adding nigericin to deplete any remaining ΔpH did not cause a further drop in fluorescence for either sample, indicating both nisin and subtilosin caused a total depletion of the ΔpH (FIGS. 26A, B) through formation of transmembrane pores.

The results show that subtilosin acts by fully depleting the transmembrane pH gradient (ΔpH) and causing an immediate efflux of intracellular ATP, but has no effect on the transmembrane electric potential (ΔΨ). The current results strongly suggest that the changes in the PMF brought about by subtilosin are due to the formation of transient pores in the cytoplasmic membrane of G. vaginalis.

### Analysis of Spermicidal Activity

### Production of Subtilosin

Subtilosin was prepared as previously described in Sutyak et al., “Isolation of the Bacillus subtilis antimicrobial peptide subtilosin from the dairy product-derived Bacillus amyloliquefaciens,” Journal of Applied Microbiology 104(4): 1067-74 (2008). To prepare a cell-free supernatant (CFS), cells were removed by centrifugation (Hermle Z400K; LabNet, Woodbridge, N.J., USA) for 25 minutes at 4500 g and 4° C. The supernatant was filter sterilized using 0.45/2m filters (Fisher, Pittsburgh, Pa., USA). The protein of interest was precipitated from the supernatant by adding 30% ammonium sulfate (w/v) while stirring overnight at 4° C. and was resuspended in 20 mL of double distilled H₂0. The column chromatography method described by Sutyak et al. was used to purify subtilosin from the CFS, producing a near-pure isolate in the 90% methanol eluate. The antimicrobial activity of all samples was confirmed by the well-diffusion assay according to the protocol of Cintas et al., “Isolation and characterization of pediocin L50, a new bacteriocin from Pediococcus acidilactici with a broad inhibitory spectrum,” Applied and Environmental Microbiology 61 (7):2643-48 (1995) with additional modifications discussed in Sutyak et al. The active fraction was concentrated to dryness using a Savant SC110 Speed Vac and UVS400 Universal Vacuum System (Savant Instruments, Farmingdale, N.Y., USA), then resuspended in 1.5 mL ddH20.

### Determination of Protein Concentration

The concentration of subtilosin in the column-purified fraction was determined using the Micro BCA Protein Assay Kit according to the manufacturer's protocol (Pierce, Rockford, Ill., USA). In brief, the assay measures the reduction of Cu²⁺ to Cu¹⁺ by colorimetric detection of Cu¹⁺ by bicinchoninic acid. Bovine serum albumin (BSA) was used to develop a standard curve with concentrations ranging from 0.5 to 20 μg/mL; the concentration of subtilosin was calculated using the R value from the trendline of the standard curve graph.

The concentration of subtilosin in the CFS was not measurable with the Micro BCA Protein Assay due to the high level of background proteins in the solvent (MRS medium). As an alternative, the protein concentration was calculated by comparing the antimicrobial activity of known concentrations of column-purified protein to equal volumes of CFS. Five two-fold dilutions were made from the stock samples of both the CFS and the column-purified fraction. Well diffusion assays were performed using 50 μL of each dilution against Micrococcus luteus ATCC 10420, which is commonly used as a reference microorganism for the determination of a bacteriocin's biological activity.

### Determination of the Presence of Weak Organic Acids

As reported previously, the concentration of lactic acid in the CFS was measured to assess its potential effects on antimicrobial activity and cell viability. Sutyak et al., “Isolation of the Bacillus subtilis antimicrobial peptide subtilosin from the dairy product-derived Bacillus amyloliquefaciens,” Journal of Applied Microbiology 104(4): 1067-74 (2008). The quantity of each form of the acid in the sample was measured using a commercially available D-lactic acid/L-lactic acid kit (Roche Boehringer, Mannheim, Germany), according to the manufacturer's instructions.

### EpiVaginal Ectocervical Tissue Model

The EpiVaginal (VEC-100) ectocervical tissue model (MatTek Corporation, Ashland, Mass., USA) was used and maintained as fully described by Dover et al., “Safety study of an antimicrobial peptide lactocin 160, produced by the vaginal Lactobacillus rhamnosus,” Infectious Diseases in Obstetrics and Gynecology 6 pages, Article ID 78248 (2007). The tissues were exposed to 83 μL of subtilosin CFS (˜136 μg/mL) for 4, 24, and 48 hours. For exposure times over 24 hours, the tissues were aerated by placing them on two metal washers (MatTek Corporation, Ashland, Mass., USA) and fed with 5 mL of the assay medium. Double-distilled water (ddH20) was used as a negative control, and was applied to cells after 6, 24, and 48 hours. A spermicidal product containing 4% Nonoxynol-9 (Ortho Options CONCEPTROL Vaginal Contraception Gel, Advanced Care Products, Skillman, N.J., USA) was used as a positive control based on its documented cytotoxic properties. A cream (Monistat-3, Ortho McNeil Pharmaceutical, Inc., Raritan, N.J., USA) containing 4% of the nontoxic, BV-active compound miconazole nitrate, was used as a negative control.

Following the designated exposure times, the MTT (3-(4, 5-Dimethylthiazol-2-yl)-2, 5-diphenyltetrazolium bromide) assay was used to determine overall cell viability. The data were used to approximate an effective time (ET) that would reduce cell viability to 50% (ET-50).

### MTT Viability Assay

The MTT assay was carried out according to the protocol outlined by Dover et al. Briefly, the viability of ectocervical cells after exposure to subtilosin was measured as a direct proportion of the breakdown of the yellow compound tetrazolium to the purple compound formazan, since only living cells can cause this reaction to occur. Tissues were exposed to subtilosin and the two controls for several designated time points; at the conclusion of each, the liquid in the plate wells was combined with the liquid from the tissue inserts. This mixture was then assayed spectrophotometrically using a 96 well-plate reader (MRX revelation, Dynex Technologies, Va, USA) to determine the level of tetrazolium degradation.

The viability (%) of the treated tissue inserts was calculated according to an equation provided by the manufacturer: % viability=OD₅₇₀ (treated tissue)/OD₅₇₀ (negative control tissue). The exposure time that reduced tissue viability by 50% (ET-50) was calculated according to [V=a+b*log(t)] described by Ayehunie et al., “Organotypic human vaginal-ectocervical tissue model for irritation studies of spermicides, microbicides, and feminine-care products,” Toxicology in Vitro 20(5):689-98 (2006), where V=% viability, t=time in minutes, and “a” and “b” are constants representing the viability data from the time points preceding and following 50% viability. On the whole, there is a direct relationship between the length of the ET-50 and the toxicity of the tested application (i.e., a shorter ET-50 corresponds to a more harmful compound).

### Semen Sample Collection and Analysis

The CFS gathered from a B. amyloliquefaciens culture was used to test the effect of subtilosin exposure on the motility of human spermatozoa. Initially, the CFS was diluted with normal saline (0.9%) so that 200 μL of the final material was equivalent to 50 μL, 100 μL, or 200 μL of undiluted CFS.

Two semen samples were collected on the day of experimentation. Each sample was collected by self-masturbation in a polypropylene specimen container (Fisher) prior to transport to the laboratory. Within 1 hour of collection, the samples were pooled. Total sperm count was calculated using bright field light microscopy (Olympus BX50; 400×) after dilution (1:50) of the semen in normal saline. The initial percentage of motile sperm was calculated prior to testing with a neubauer hemacytometer. The determination of motile sperm % was made using randomly Selected field views (400×) from a count of between 104-201 cells. Any visibly moving spermatozoa (directional or stationary) were counted as motile cells.

The percentage of forward progressing spermatozoa was subjectively determined based on the assumption that 70% of the sperm in a normal sample would behave in such a manner The samples used in this experimentation fell into such a “normal” category.

Treatment of Spermatozoa with Subtilosin

A modified Sander-Cramer test was used to determine the effect of column-purified subtilosin on human spermatozoa motility. This measured the effect of subtilosin after 30-second exposure times of 5 volumes (200 μL) of the solution at each dilution (25% and 50% in normal saline, and 100%) with one volume (40 μL) of whole semen. The motilities of cells from random high-magnification fields (400×) of the sample were determined in duplicate as described above.

### Data Analysis

The % motility data were arcsine transformed prior to further examination. StatMost32 (version 4.1) statistical software (DataMost Corporation, Sandy, Utah, USA) was used to calculate all statistical parameters. The % values of motility were presented as averages and 90% confidence limits. Any differences between treatment groups were assessed by the Newman-Keuls multiple range test. Differences were deemed significant at the 0.05 level of confidence.

### Determination of Protein Concentration

The concentration of subtilosin in the column-purified sample was estimated at 135.7 μg/mL. The CFS and column-purified sample produced identical zones of inhibition at each dilution (data not shown); therefore, the concentrations of protein in both solutions were assumed to be equivalent. While it is improbable that a 100% yield would be attained from column chromatography, previous work has shown that protein concentrations can be precisely calculated based on the comparisons we conducted. Due to the difficulty in measuring the CFS protein concentration via other assays, the chosen method was deemed the most accurate and reproducible.

### Cell Viability % and ET-50 Values

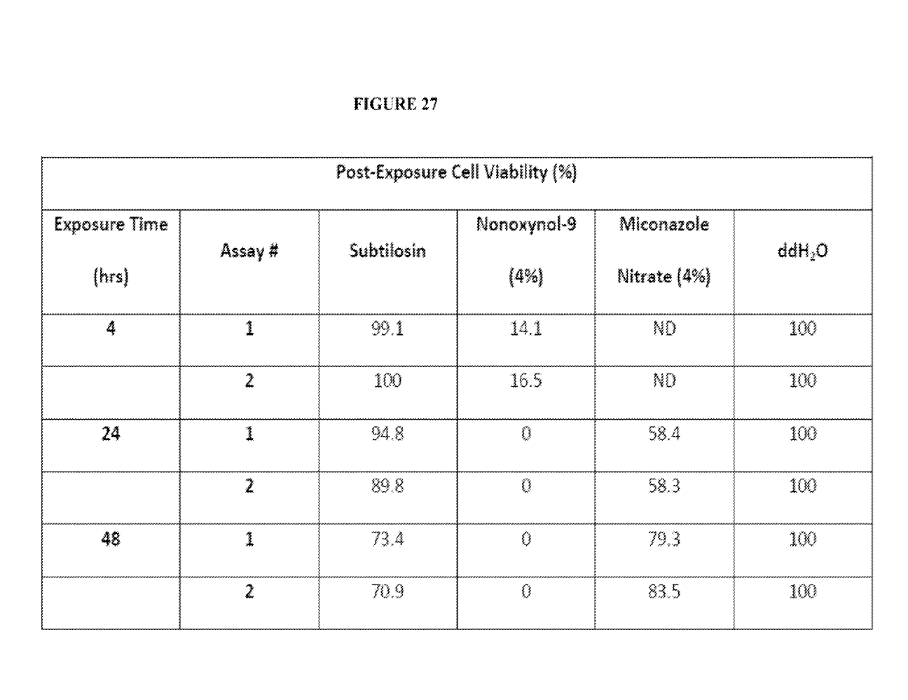

After 48 hours of exposure to subtilosin, the EpiVaginal ectocervical tissues retained a high level of viability compared to the positive control, Nonoxynol-9, and the negative control, miconazole nitrate (FIG. 27). Due to the lack of toxicity of the antimicrobial, the ET-50 value for subtilosin could not be established since the total cell viability did not drop below 50% at any of the given time points. However, a projection of the ET-50 value is possible by an extrapolation of the data. Data presented in Table 1 can be fit to a curve described by Ln(V)=a+bt², where a=4.605995356 and b=−0.00014151 (coefficient of determination, or r²,=0.9998), from which the ET-50 is estimated at 70 hours.

### Quantitative Observations of Motile Spermatozoa

Subtilosin reduces human sperm motility in a dose-dependent manner (FIG. 16). The percentage of motile spermatozoa in pooled whole semen was determined 30 seconds after mixing with subtilosin A, at different final concentrations, as indicated. All data were adjusted to a normal control motility of 70% and subjected to arcsine transformation before further analysis. Values are expressed as average % motility. Error bars are 90% confidence limits. The motility of the treated spermatozoa ranged from 0 to 88% of control motility levels over the fourfold range of subtilosin concentrations. All of the subtilosin concentrations tested reduced motility compared to the control samples. The differences in the proportion of motile spermatozoa in all samples (28.3, 56.7, and 113.3 μg/mL protein equivalents) were found to be significant (P<0.05) according to the Newman-Keuls multiple range test. TableCurve 2D (ver 5.0) curve-fitting software (SPSS Scientific Software, Chicago, Ill., USA) was used to fit the data to a dose-response curve described by Ln. (% Motility)=a+b [Subtilosin A]₃, where a=4.20781; b=−2.5814e−06; and [subtilosin A] is expressed as μg/mL protein equivalents. The curve had a coefficient of determination (r²)=0.9959. The IC₅₀ value, or the amount of subtilosin required to reduce the motility of spermatozoa in whole semen by 50%, was calculated to be 64.5 μg/mL.

### Semiquantitative Observations of Spermatozoa: Forward Progression

Similar to motility, forward progression of spermatozoa is reduced in a dose-dependent fashion by subtilosin. In control samples, 70% of sperm exhibited forward progression; in the presence of 50 μL subtilosin this decreased to 50-70%, while 100 μL caused a decline to only 10% forward progression. All forward progression was eliminated after treatment with 200 μL subtilosin, with most sperm tails becoming coiled.

Subtilosin was found to significantly reduce the motility of human spermatozoa in a concentration-dependent manner for all concentrations tested. The effect of subtilosin on the forward progression of spermatozoa was also observed to be a dose-dependent interaction. Serial dilutions showed a steady decline in forward progression, with all progression halted at the highest concentration tested. It was also noted that at the highest concentration, the tails of the sperm cells were curved or coiled, indicating the cells were damaged beyond a simple restriction of movement. Coiling of the cells is considered to be a sperm abnormality, and may indicate damage to the plasma membrane. Tail coiling has been observed after in vitro exposure of monkey spermatozoa to methyl mercury.

Subtilosin in Combination with Other Natural Antimicrobials

### Bacterial Strains and Growth Conditions

Gardnerella vaginalis ATCC 14018 cultures were grown anaerobically in BHI broth(Difco, Sparks, Md.)+3% horse serum (JRH Biosciences, Lenexa, Kans.) at 37° C. without shaking. B. amyloliquefaciens cultures were grown overnight in MRS broth (Difco) at 37° C. without shaking. Initial cultures of both organisms were subcultured multiple times before use. For all experiments, G. vaginalis was grown overnight to an approximate cellconcentration of 108 CFU/mL, then diluted 100-fold in growth medium for a workingconcentration of 106 CFU/mL. Stock cultures of both organisms were kept at −80° C. intheir appropriate growth medium supplemented with 15% v/v glycerol. Preparation of Antimicrobial Solutions

The partially purified preparation of subtilosin was prepared as described above. Sterile Lauricidin® (glycerol monolaurate) was a gift from Dr. Alla Aroutcheva of Rush Medical Center, Chicago, Ill. A 2 mg/mL stock solution of glycerol monolaurate was prepared in BHI+3% horse serum broth pre-warmed to 37° C. MIRENAT-CF was a gift from Vedeqsa Corp. (Barcelona, Spain), and contained 1 mg/mL lauric arginate (N^{α}-lauroyl-L-arginine ethyl ester monohydrochloride, LAE). A stock solution containing 25% E-poly-L-lysine (250 mg/mL) was a gift from Chisso America, Inc. (Lot #2090501; Rye, N.Y.). A solid stock supply of zinc lactate (Puramex Zn) was a gift from Purac America, Inc. (Lot #0807000376; Lincolnshire, Ill.). A 5.45 mg/mL stock solution of zinc lactate was made using ddH₂O. All antimicrobial solutions were filter-sterilized using a 0.45 μm filter (Nalgene, Rochester, N.Y.) prior to use.

### Determination of Minimal Inhibitory Concentrations (MICs)

The ability of each antimicrobial to individually inhibit G. vaginalis growth was determined using the broth microdilution method as per Amrouche, T., Sutyak, et al., “Antibacterial activity of subtilosin alone and combined with curcumin, poly-lysine, and zinc lactate against Listeria monocytogenes strains,” Probiotics Antimicrob Prot. doi 10.1007/s12602-010-9042-7 (2010), with slight modifications. From the stock solutions, 10-fold serial dilutions of each antimicrobial (subtilosin: 230-0.023 ug/mL; glycerol monolaurate: 200-0.02 ug/mL; lauric arginate: 10,000-10 ug/mL; poly-lysine: 25,000-25 ug/mL; zinc lactate: 5450-0.545 ug/mL) in the proper diluent. G. vaginalis cells were grown overnight and prepared as previously described. A sterile, 96-well microplate (Corning, Inc., Corning, N.Y.) was prepared by adding the serial dilutions of antimicrobials in horizontal rows, descending from highest concentration to lowest concentration tested. The antimicrobials were tested in 20 μL increments (0-100 μL), with each volume tested in duplicate. The volume of each well was raised to 100 μL total with the addition of sterile ddH₂O, and the contents of each well were mixed by gentle pipetting. One hundred μL of G. vaginalis cells were added to each well; wells containing cells alone, antimicrobial alone, water alone, and growth medium alone were used as controls. Fifty μL of sterile mineral oil was pipetted onto the top of each well to form an airtight seal that would allow for anaerobic growth of the G. vaginalis cells. Each plate was then transferred into a Coy Type C Anaerobic Chamber (Coy Laboratory Products, Inc., Grass Lake, Mich.) and placed in a Bio-Rad Model 550 Microplate Reader (Bio-Rad Life Sciences, Hercules, Calif.). The turbidity of each well was recorded at 595 nm every 30 min. for 48 hrs at 37° C. In order to prevent mixing of the mineral oil seal with the contents of each well, the plate was not shaken prior to each measurement. Data was gathered and analyzed using Microplate Manager (version 5.1.2) software (Bio-Rad). The lowest concentration of each antimicrobial that showed no increase in optical density (no bacterial growth) was designated as the MIC. Each assay was performed at least twice in duplicate.

### Checkerboard Assay

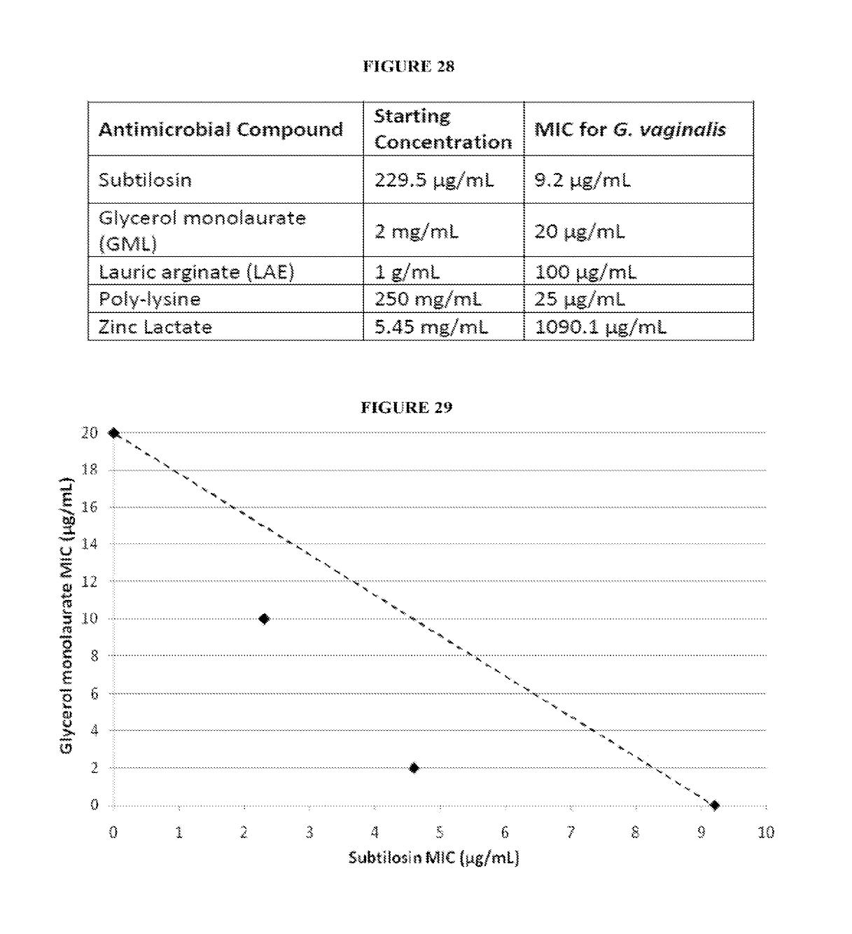

The interaction between subtilosin and the chosen antimicrobials was tested via a “checkerboard” assay that allowed for testing of multiple antimicrobials at various concentrations at the same time. The assays were performed according to Badaoui Najjar, et al., “Epsilon-poly-L-lysine and nisin A act synergistically against Gram-positive food-borne pathogens Bacillus cereus and Listeria monocytogenes,” Lett. Appl. Microbiol. 45: 13-18 (2007). with the following modifications. In each experiment, a sterile 96-well microplate (Corning) was prepared so that subtilosin (horizontal rows) would be combined with one of the chosen antimicrobials (vertical columns). Using a stock solution of a 10-fold higher concentration than its respective MIC, each compound was aliquoted into the appropriate row or column. Each plate was designed to test concentrations directly above, equal to, and, particularly, below that of the individual MIC of each antimicrobial (FIG. 28). The volume of each well was raised to 100 μL using sterile ddH₂O. G. vaginalis were grown overnight and prepared as previously described; 100 μL of this preparation was added to each well. The first row and column of the microplate served as controls (no antimicrobials), as did a row of water alone and growth medium alone. Fifty μL of sterile mineral oil was pipette onto the top of each well to ensure anaerobic conditions. Each plate was run using the same equipment and under the same conditions as described in the previous section. Each MIC assay tested a wide range of concentrations for each compound, and was conducted at least twice in duplicate. All assays conducted resulted in identical results for all substances (no standard deviation).

### Graphical Presentation of the Data

The kinetic growth curve data from all assays was analyzed using Microsoft Excel 2007 (Microsoft, Redmond, Wash.). Isobolograms were created for each synergy assay as a way to visualize the presence of synergy, additive effect, or antagonism. In an isobologram, the x- and y-axes represent the concentrations of each antimicrobial; the MIC of each substance is then plotted on the graph, and the two points are joined by a line. The mixed concentrations of antimicrobials that caused complete inhibition of microbial growth are then plotted on the graph. Points that fall below the line indicate synergy, points on the line show an additive effect, and points above the line demonstrate antagonism. (Chou, T.-C., “Theoretical basis, experimental design, and computerized simulation of synergism and antagonism in drug combination studies,” Pharmacol Rev. 58: 621-81 (2006)).

### Determination of MICs

The MIC of subtilosin, GML, LAE, poly-lysine, and zinc lactate against G. vaginalis was tested by the broth microdilution method in BHI broth supplemented with 3% horse serum. As seen in FIG. 28, all of the tested substances were able to completely inhibit the growth of the selected vaginal pathogen Subtilosin proved to be quite effective with an MIC of only 9.2 μg/mL, while GML and poly-lysine had MICs of 20 μg/mL and 25 μg/mL, respectively. The MIC of GML is supported by the findings of Strandberg, K. L., et al., “Glycerol monolaurate inhibits Candida and Gardnerella vaginalis in vitro and in vivo but not Lactobacillus,” Antimicrob Agents Chemother. 54: 597-601 (2010), who demonstrated that GML had an MIC of 10 μg/mL against a clinical isolate of G. vaginalis. At 1.0901 mg/mL, the MIC of zinc lactate was 40-fold higher than that of the other tested compounds. As previously stated, all MIC assays were run at least two times in duplicate. The results for each compound did not deviate between assays, despite the extensive range of tested concentrations; thus, there was no standard deviation recorded for these results (FIG. 28).

### Determination of Synergy Between Antimicrobial Substances

Once the individual MICs of all the chosen compounds were calculated, a checkerboard assay was performed using subtilosin in combination with one other substance. Each assay was designed to test a wide range of concentrations, beginning with one slightly above that of each compound's individual MIC and decreasing in a serial manner to a zero concentration (negative control). Combinations of concentrations below each of the MIC levels that caused complete inhibition of microbial growth were analyzed with isobolograms to determine the presence of synergy, additive effect, or antagonism.

### Interaction Between Subtilosin and Glycerol Monolaurate (GML)

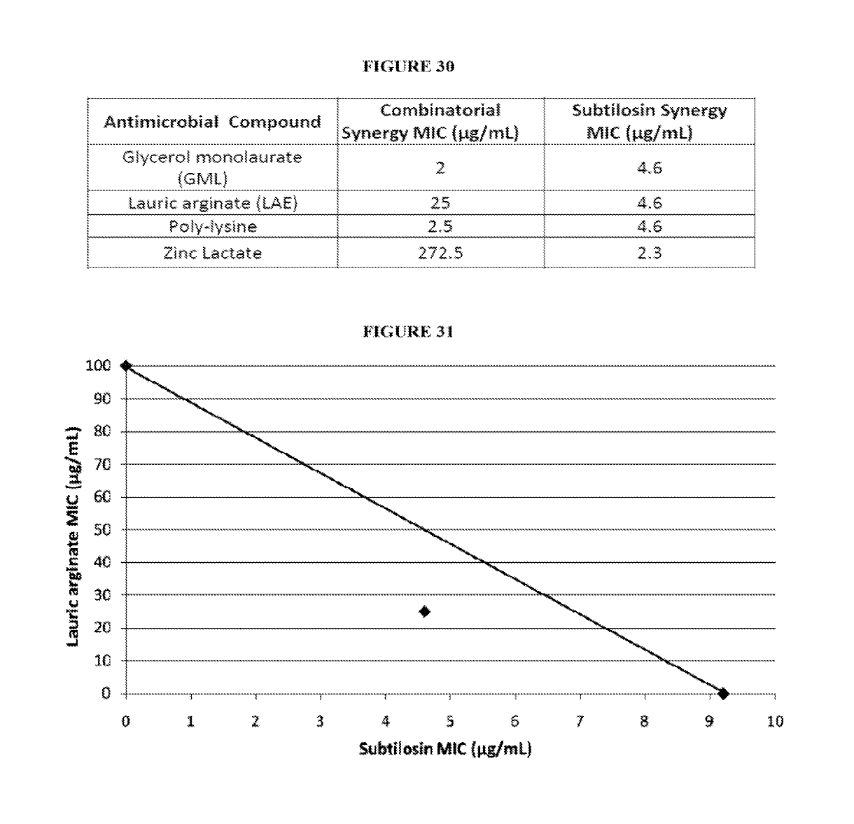

Since GML has demonstrated antimicrobial activity against the BV-associated pathogen G. vaginalis, it was the first substance tested for synergy with our target peptide, subtilosin. To visualize synergy between combinations of the two compounds, an isobologram was constructed by plotting the individual MICs of subtilosin on the x-axis and GML on the y-axis and connecting the two points (FIG. 29). From the checkerboard assay, the lowest combined concentrations of subtilosin and GML that caused total growth inhibition of G. vaginalis were 4.6 and 2 μg/mL, respectively (FIG. 30). When used in combination, there was a two-fold reduction in subtilosin's MIC and a four-fold reduction in GML's MIC. The point representing these two concentrations was added to the isobologram and falls well below the trendline, indicating synergy. While the concentration combinations of 2.3 μg/mL subtilosin and 10 μg/mL GML also caused complete inhibition of G. vaginalis growth, the corresponding graph point fell closer to the trendline, indicating weaker synergy (FIG. 29).

### Interaction Between Subtilosin and Lauric Arginate (LAE)

The second natural antimicrobial, lauric arginate, has previously been shown to synergize with the Lactobacillus rhamnosus-produced bacteriocin lactocin 160 against G. vaginalis (Y. Turovskiy, personal communication). As described for GML, its potential synergy with subtilosin was assessed and an isobologram was constructed using the individual MICs of subtilosin and LAE (FIG. 31). The checkerboard assay showed the lowest concentration combination of subtilosin and LAE that caused complete inhibition of G. vaginalis growth to be 4.6 μg/mL and 25 μg/mL, respectively (FIG. 30). This combination caused a two-fold decrease in subtilosin's individual MIC and a four-fold reduction in LAE's MIC. When plotted on the isobologram, the point representing these two concentrations also falls below the trendline, indicating synergy between the two compounds (FIG. 31).

### Interaction Between Subtilosin and E-poly-L-lysine

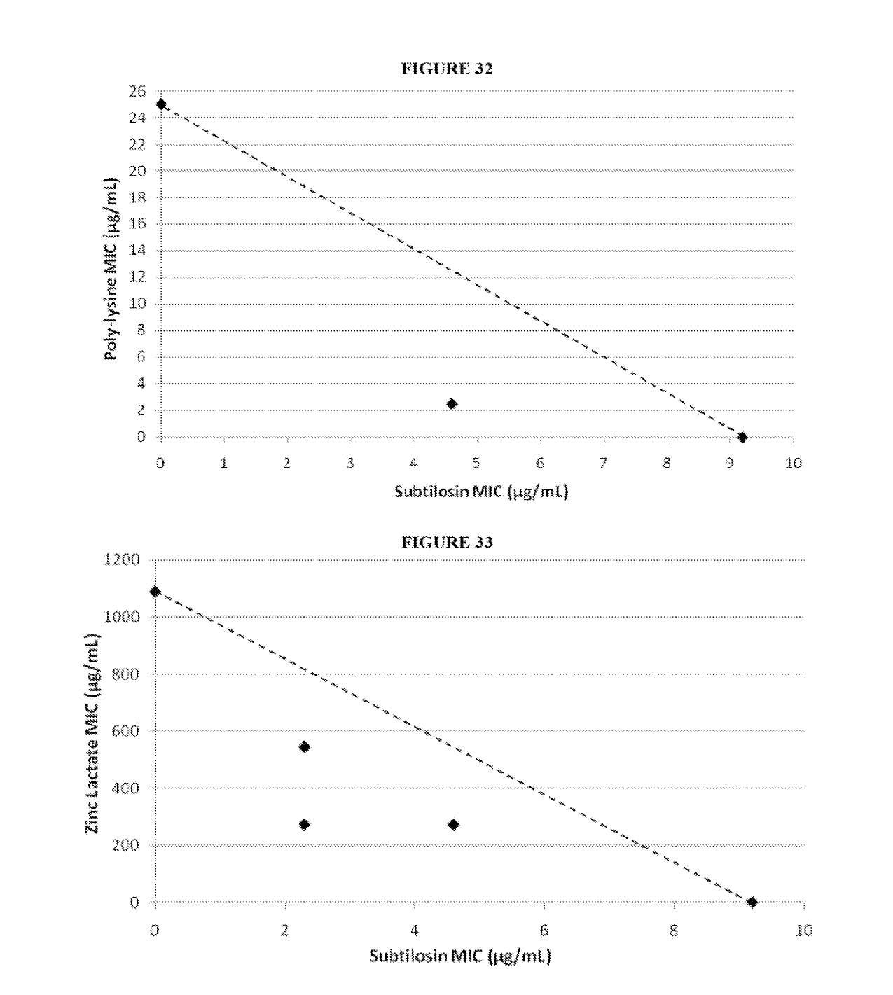

An isobologram was constructed using the individual MICs of subtilosin and poly-lysine (FIG. 32). The checkerboard assay exhibited the lowest concentration combination of subtilosin and poly-lysine to completely inhibit G. vaginalis growth as 4.6 μg/mL and 2.5 μg/mL, respectively (FIG. 30). This combination caused a two-fold decrease in subtilosin's individual MIC and a significant ten-fold reduction in poly-lysine's MIC. When plotted on the isobologram, the point representing these two concentrations also falls below the trendline, indicating synergy between the two compounds (FIG. 32).

### Interaction Between Subtilosin and Zinc Lactate

An isobologram was constructed using the individual MICs of subtilosin and zinc lactate (FIG. 33). The checkerboard assay demonstrated that the combination of the lowest concentrations of subtilosin and zinc lactate that fully prevented G. vaginalis growth were 2.3 μg/mL and 272.5 μg/mL, respectively (FIG. 30). This combination caused a four-fold decrease in subtilosin's individual MIC and a five-fold decrease in zinc lactate's MIC. When plotted on the isobologram, the point representing these two concentrations falls below the trendline, indicating synergy between the two compounds (FIG. 33). While two other concentration combinations (2.3 μg/mL subtilosin and 545 μg/mL poly-lysine; 4.6 μg/mL subtilosin and 272.5 μg/mL zinc lactate) also caused complete inhibition of G. vaginalis growth, the corresponding graph points were closer to the trendline, indicating weaker synergy (FIG. 33).

### Discussion

The antimicrobial activity of subtilosin and four natural antimicrobials were investigated alone and in combination against the BV-associated pathogen G. vaginalis. A checkerboard assay was utilized to study multiple concentrations of subtilosin and another antimicrobial compound for the presence of synergy, additive effect, or antagonism against the target microorganism. Individually, subtilosin had the lowest MIC against subtilosin at 9.2 μg/mL, although GML, LAE, and poly-lysine also had MICs in the μg/mL range. On its own, zinc lactate was shown to be less effective against G. vaginalis with an MIC of slightly over 1 mg/mL (FIG. 28). However, when each of the four compounds were tested in combination with subtilosin, there was a dramatic reduction in their MIC. Both GML and LAE's MICs were reduced four-fold, while subtilosin's MIC decreased by half. The ten-fold drop in poly-lysine's MIC was the most significant change, while zinc lactate's relatively high individual MIC was decreased fivefold (FIG. 30). As seen in each of the isobolograms (FIGS. 29 and 31-33), the points representing the combinatorial MICs of subtilosin and the secondary antimicrobial all fall well below the trendlines connecting the points depicting each compound's individual MIC. As such, it is apparent that subtilosin synergizes with all of the tested antimicrobials.

What is claimed is:

1. A topical composition comprising an antimicrobially and/or spermicidally effective amount of subtilosin incorporated into a pharmaceutically acceptable aqueous solution, non-aqueous solution, nanofiber, hydrogel, gel, nanogel, suspension, ointment, jelly, insert, suppository, sponge, salve, cream, foam, foaming tablet, or douche.

2. The topical composition of claim 17, wherein said hydrogel is a multifunctional polyethylene glycol-based hydrogel comprising a multi-arm polyethylene glycol cross-linking unit covalently bound to at least four multi-arm polyethylene glycol nanocarrier units, wherein each nanocarrier unit comprises an agent coupled to the nanocarrier unit and each agent is selected from the group consisting of pH-lowering agents, bioadhesion agents, microbiocidal agents, spermicidal agents, and agents that inhibit free and cell-associated HIV binding, provided that each nanocarrier unit comprises a different agent.

3. The composition of claim 2 further comprising an additional antimicrobial agent.

4. The composition of claim 3, wherein the additional antimicrobial agent is selected from the group consisting of glycerol monolaurate, lauric arginate, polylysine, and zinc lactate.

5. The composition of claim 2 comprising a bacterial vaginosis-prophylactic combination of subtilosin and further comprising polylysine, wherein the subtilosin and polylysine are present in amounts such that the combination provides reduced minimum inhibitory concentration values versus Gardnerella vaginalis when compared with the minimum inhibitory concentration of subtilosin alone and the minimum inhibitory concentration of polylysine alone.

6. The composition of claim 5 wherein said combination of subilosin and polylysine is synergistic versus Gardnerella vaginalis.

7. The composition of claim 5, wherein the minimum inhibitory concentration of subtilosin in the combination is about 50% of the minimum inhibitory concentration of subtilosin when not combined with polylysine.

8. The composition of claim 5, wherein the minimum inhibitory concentration of polylysine in the combination is about 10% of the minimum inhibitory concentration of polylysine when not combined with subtilosin.

9. The composition of claim 2, wherein at least two nanocarrier units comprise an agent having a different functionality.

10. The composition of claim 2, wherein at least one agent is coupled to a nanocarrier unit via a degradable bond.

11. The composition of claim 2, wherein at least one agent is coupled to a nanocarrier via a nondegradable bond.

12. The composition of claim 2 further comprising a pH-lowering agent selected from the group consisting of lactic acid, citric acid, ascorbic acid, and maleic acid.

13. The composition of claim 2 comprising a pH-lowering agent encapsulated in a carrier.

14. The composition of claim 13, wherein said carrier is from the group consisting of cyclodextrins, dendrons, dendrimers, liposomes, and PEG nanogel particles.

15. The composition of claim 2 further comprising an agent that inhibits free and cell-associated HIV binding selected from the group consisting of soluble polyanions and an RGD peptide.

16. The composition of claim 15, wherein the soluble polyanion is selected from the group consisting of dextran sulfate, cyclodextrin sulfate, and heparin.

17. The composition of claim 2 further comprising at least one nanocarrier unit noncovalently bound within the hydrogel.

18. An article comprising the composition of claim 2.

19. A method for treating bacterial vaginosis in a patient in need thereof, comprising intravaginally applying a treatment-effective amount of the composition of claim 2 to said patient.

20. A method for treating bacterial vaginosis in a patient in need thereof, comprising intravaginally applying a treatment-effective amount of the composition of claim 4 to said patient.
